(19) United States
(12) Patent Application Publication
(10) Pub. No.: US 2026/0041520 A1
(43) Pub. Date: Feb. 12, 2026

(54) Subcutaneous visualization system

(71) Applicant: Houda Hippler, Berlin (DE)

(72) Inventor: Houda Hippler, Berlin (DE)

(21) Appl. No.: 18/801,007

(22) Filed: Aug. 12, 2024

(51) Int. Cl. A61B 90/00; A61M 39/02

(52) U.S. Cl. CPC A61B 90/37; A61M 39/0247; A61B 2090/3616; A61B 2090/365; A61M 2205/507

(57) ABSTRACT

The present subject matter relates to a visualization system, that employs advanced imaging technologies and see-through smart devices, in order to assist the user, to visualise, localise and detect foreign entities within a biological entity, by directly providing real-time processed information to a user. This system is designed/created to enhance the accuracy and efficiency of medical/research procedures/investigations, by clearly differentiating in between, a biological entity (including, all components, parts, portions of the biological entity), and, any kind of foreign entities disposed within the said biological entity. Further, the innovative system enables simple non-invasive method of detecting and visualising, a foreign entity that is disposed within a biological entity.

### FIELD OF THE INVENTION

The present subject matter, relates to a method and system of subcutaneous/intradermal/visceral visualization. More specifically, for subcutaneous visualization, that is comprising of but is not limited to, the utilisation of technologies such as, see-through smart devices, augmented-reality interfaces, EM-frequency transmission-detection, digital visualization, graphical data processing, object identification, etc., primarily in order to provide visual-assistance to a user, by helping them locate and clearly visualize the features of a subcutaneous foreign entity, that is disposed inside a biological entity of interest.

### BACKGROUND OF THE INVENTION

In medical procedures, accurate visualization is critical for ensuring successful outcomes, especially when visualising foreign entities, such as implants, diseases, injuries, toxins, and many other objects that are alien to the body, such as, bullets, shrapnel, glass shards, plastic, etc. Traditional imaging methods while effective, often lack true real-time capabilities and sometimes it is absolutely cumbersome to use them due to their non-portable nature, especially during surgical procedures. There is a need within the medical field, for a more integrated, portable and user-friendly solution, that provides real-time visualization, localization, detection of different types of both biological and foreign entities, with a high-precision and clarity. Further, there is a definite lack of any system, that provides a clear-differentiation between a foreign entity and a biological entity that encompasses the foreign entity inside/within itself.

Specifically, subcutaneous/visceral implants are commonly used in various medical applications, including drug delivery systems, monitoring devices, Port-a-Caths, pacemakers, prosthetic implants, etc. Here, there is no prior art, that enables a user/medical practitioner to visually locate and identify these various subcutaneous/intradermal/visceral implants, inside the body of a biological entity, without cutting open a part of the said biological entity, or, alternatively by utilising specifically modified-implants. Further, there is no prior art that enables a user/medical practitioner to visually locate and identify the various injuries, such as but is not limited to, depth of the cut, cell lysis area, tissue damage, internal haemorrhaging, muscle tear/damage, bone crack/damage, lacerations, contusions, internal injuries, etc., without cutting open any part of the said biological entity, in high detail. Furthermore there is no prior art, that enables a user/medical practitioner to visually locate and identify the various diseases/ailments, such as but is not limited to, virus, bacteria, fungus, cancer, tumour, cyst, blood-clot, or other known diseases within the field, that are present inside the body of a biological entity, without cutting open any part of the said biological entity, time-consuming/arduous X-Ray/CT/MRI scans, or, inserting any type of probe into the body of the biological entity. Moreover, there is no prior art that enables a user/medical practitioner to visually locate and identify the various toxins/poisonous substances, such as but is not limited to, enterotoxins, phycotoxins, phytotoxins, mycotoxins, zootoxins (like poisons, venoms, etc.), etc., inside the body of a biological entity, without cutting open any part of the said biological entity, or, inserting any type of probe into the body of the biological entity. Lastly, there is no prior art that enables a user/medical practitioner to visually locate and identify other various alien entities, such as but is not limited to, bullets, shrapnel, glass shards, etc., and especially their specific trajectory within the body of a biological entity, without cutting open a part of the said biological entity, time-consuming/arduous X-Ray/CT/MRI scans, or, inserting any type of probe into the body of the biological entity.

Further, many of these foreign entities often require precise localization in order for patient safety and more importantly, to provide ease and comfort in any procedure with respect to these foreign entities, such as for instance, implants. Traditional methods for detecting and visualizing foreign entities like implants, either involve invasive procedures, or, imaging techniques such as X-rays, CT, or MRI based scanning, and/or even manual palpation (such as for instance when the implant is a port-a-cath), all of which can be uncomfortable for patients, cause unnecessary delays, may even pose risks of infection or types of other complications and these methods may not even always provide accurate results, as intended. Some present-day methods for capturing the surgical/medical sites of such scenarios often use headsets that may include a projection system for displaying a virtual view of internal bones, veins, muscles and organs in an eyepiece. Conventionally, these systems use headsets mounted with cameras and viewing screens to determine the location of veins (intravenous access) for drawing blood or injecting medicines into these veins. However, these methods do not provide any way of clearly detecting foreign entities (specifically, unmodified implants, meaning that the implants, do not have any reflectors, RFID chips, etc. for providing any assistance in detection/localization), within the body of any biological entity. Further, these completely fail to clearly differentiate and provide a visualization of these foreign entities.

Recent advancements in augmented reality (AR) and smart device technology offer new opportunities for non-invasive and real-time visualization of subcutaneous foreign entities. However, existing solutions often lack the integration of sophisticated detection technologies while providing user-friendly interfaces, limiting their effectiveness in clinical/small-scale settings. Further, few of these previous solutions employ modified subcutaneous implants, that are either modified by adding sensors/arrays, reflectors, RFID, magnetic materials, transmitters, etc. to make them detectable, however these additions have no other purpose, except other than to, ease the detection, orientation and localization of these subcutaneous implants, once they are inside the body of the biological entity. Hence, there is a need for a system that can accurately detects, visualizes and differentiates, between a biological entity and an unmodified foreign entity in real-time, by providing the user/medical-practitioners with the necessary information to perform procedures/investigations with a greater degree of precision and ease. The conventional systems typically rely on AR systems with a frame, lens, and multiple cameras configured to record real-time images, which may include parts of the subject's body or surgical instruments. However, these systems do not detect unmodified passive implants like, port-a-Cath, pace-maker, knee-implant, hip-implant, cardiac implant, dental implant, etc. inside the body of a biological entity, or, a patient. Further, these systems definitely are not capable of detecting/localizing/identifying, any other type of unmodified foreign entity, either.

However, these previous market available products lack the capability to locate and visualize, different types of implants within the body. Though the conventional inventions can identify the location and orientation of the implants having LED lights or other electronic identifiers, such as for example, a centrally located radio-frequency identification (RFID) tag. Moreover, these past solutions fail to identify differing shapes, sizes, textures, materials, and/or orientations of the foreign entities or, more specifically, for example, an unmodified subcutaneous implant, or, even any other unmodified foreign entity. This limitation arises from the digital visualization techniques used by past innovators, which may not be sophisticated enough to capture the detailed geometries and spatial configurations of different types/kinds of the unmodified foreign entities. For instance, an implant's position might shift slightly after insertion, or its shape might vary, from one patient to another, making it a challenging task for the conventional systems to provide precise and reliable visual information in real-time, with respect to, an unmodified foreign-entity, or, especially a customised subcutaneous/intradermal/visceral implant. Lastly, some of these past systems utilise the visualization technology for vein determination, using infrared light for illumination. However, these methods are not optimized for accurately detecting foreign entities, let alone localization, visualization and orientation. As these traditional systems for detecting subcutaneous implants, often use only infrared light-sources and these systems are only directed towards effectively locating veins, but have several significant limitations, when it comes to identifying foreign entities having various kinds of materials with high-precision. Specifically, these systems struggle to detect materials, such as for example, silicone, metals, ceramics, plastics, etc. with high-precision. While silicone is one of most commonly used materials within various types of medical implants, due to the wide-range of biocompatibility and flexibility, that is provided by silicone, at least the prospect of detecting silicone with a high degree of accuracy, is an imperative requirement within the domain.

Other current market solutions, for detecting, visualizing implants, often require manual operation engaging at least one hand of the user, which can be cumbersome and less efficient for medical practitioners. The non-handsfree nature, of these devices means that, healthcare providers need to use their hands to manipulate the equipment, potentially leading to a less sterile environment and increased risk of infection. Additionally, this can slow down the process and reduce the precision with which the foreign entities are located and visualized.

Many existing systems lack wireless connectivity and rely on wired connections, which can restrict mobility and flexibility during medical procedures. The absence of a detachable detection ensemble, further limits the practicality of these past systems. A wireless portable detachable detection unit would offer greater convenience, by allowing practitioners to move freely and position the detection unit, as needed without being hindered by cables.

Currently, no available subcutaneous detection methods utilize augmented reality (AR) glasses to visualize the inner/visceral structures of any biological entity. Essentially, no AR glasses in the market provides a real-time, see-through view of the biological entity's internal layers/regions, enabling medical practitioners to visualize the exact location and orientation of any kind of foreign entities, without having to perform any kind of invasive procedures on the biological entity. Further, none of the current solutions provide a see-through device that is not bulky/awkward in appearance, this kind of appearance makes the biological entities/patients, especially children and other timid beings, feel fear/alarm after seeing a user wearing such a see-through smart device. Furthermore, no current solution provides an option, where the user may swap the type of detection/identification technique, without having to also change the visualizing see-through smart device. Moreover, other see-through smart devices, fail to corelate and provide a collated visualization data of a past X-ray, CT scan, spectroscopy, ultrasound, etc., with respect to the various portions of the 3-dimensional body of the biological entity in front of the see-through device, as a way for visualizing any kind of past 3D-scan data, in real-time.

None of the existing technologies in the prior art, incorporates AR glasses as a see-through smart device for detecting foreign entities. As a result, medical practitioners often need to rely on separate, cumbersome devices to achieve similar functionality. This in-turn may lead to, inefficiencies, unnecessary delays and increased complexity during medical procedures. Integrating AR glasses into the detection system, would streamline the process by providing a more seamless and user-friendly experience for the users, medical/practitioners, doctors, surgeons, nurses, researchers, investigators and other healthcare-providers.

Ultimately, existing solutions in the market are limited in terms of their accuracy, ease of use, and real-time capabilities. There is a need for a system that integrates advanced detection technologies with user-friendly, see-through smart devices to provide seamless and accurate visualization, of subcutaneous foreign entities.

The purpose of the present subject matter presented below, is particularly to provide a simple, economic, swift and efficient solution to the all the above described drawbacks and short-comings within the art, in order to, at least partially overcome most of the above-mentioned disadvantages.

### SUMMARY OF THE INVENTION

The present subject matter relates to a visualization system, that employs advanced imaging technologies and see-through smart devices, in order to assist the user, to visualise, localise and detect foreign entities within a biological entity, by directly providing real-time processed information to a user. This system is designed/created to enhance the accuracy and efficiency of medical/research procedures/investigations, by clearly differentiating in between, a biological entity (including, all components, parts, portions of the biological entity), and, any kind of foreign entities disposed within the said biological entity. Further, the innovative system enables simple non-invasive method of detecting and visualising, a foreign entity that is inside a biological entity.

The core component of this system is a see-through smart device, that is very similar to, augmented reality (AR) glasses, headsets, smart contact lenses, smart-phone, or, other similar AR display devices. This device essentially, presents the processed information by overlaying it, onto the user's field-of-view, allowing for seamless integration of real-time data onto the user's natural vision.

In one embodiment of the visualization system, it has an ability to provide detailed information regarding the precise location/position, shape, size, texture, material, and orientation of the foreign entities, as well as, a clear differentiation between foreign entities and the biological entity.

In some embodiments of the visualization system, the biological entities are living beings such as mammals, fish, reptiles, avians, amphibians, plants, insects, molluscs, annelids, etc. These biological entities generally contain several biological components/parts/portions, such as but is not limited to, arteries, veins, bones, muscles, skin, cartilages, ligaments, tendons, blood cells, neurons, immune cells, hair, tissues, organs, phloem, xylem, cambium, pith, pericycle, endodermis, cortex, mesophyll, etc.

In some embodiments of the visualization system, the foreign entities are the entities which are not naturally found inside living beings. These foreign entities are made up of any diverse materials such as, but is not limited to, silicon, plastic, metals, composites, combinations, etc. Further in other embodiments of the present subject matter, the foreign entities are entities, such as but is not limited to, medical implants, pathological entities, traumatological entities, toxicological entities, other alien objects, etc. Furthermore, in some other embodiments of the present subject matter, the system is designed to detect a variety of medical implants, including but is not limited to, port-a-cath, knee implants, hip implants, cardiac implant, dental implants, etc. Further, the system allows a user to insert a syringe-needle into the port-a-cath in a single attempt, by assisting them with the location and orientation of the port-a-cath.

Further, the system includes at least one transmitter-receiver unit, that has a specific-bearing that is dependent upon the type of visualization architecture being implemented. Further, the transmitter-receiver unit is equipped with a signal processing module that filters, amplifies, and converts signals, to enhance the detection accuracy of all types of foreign entities. The transmitter-receiver unit, operates within various frequency ranges, including but is not limited to, Ultrasonic, Ultrasound, Infrared, Near-Infrared, MRI, MWT, fluoroscopy, PET, endoscopy, arthroscopy, angiography, lymphangiography, mammography, venography, duplex ultrasound, elastography, OCT, EIT, photoacoustic imaging, sonography/radiology, ESI, spectroscopy, ultraviolet, modulated frequency, pulsed frequency and a Time-varying frequency, ensuring versatile application during various different medical scenarios.

The specific-bearing of the transmitter-receiver unit, is either the see-through smart device itself, or, an additional companion device. Further, in some embodiments of the present subject matter, this companion device is detachably attached to the see-through smart device. Furthermore, the companion device is capable of maintaining communication with the see-through smart device, even when detached. Moreover, in other embodiments of the present subject matter, the see-through smart device, has a plurality of companion devices, that are detachably attached to the see-through smart device, at various predefined locations on the see-through smart device, in order to gain the most optimal perspective.

Specifically, the transmitter-receiver unit is calibrated to detect foreign entities with varying sizes, shapes, texture, material, orientation and/or materials. Further, in some embodiments of the present subject matter, the transmitter-receiver unit operates in multiple modes based on the scenario, including but is not limited to, continuous scan mode, pulse mode, etc., in order to optimize the consumption of power and detection performance.

To further improve the system's functionality/accuracy, the see-through smart device also consists of a camera, in some embodiments of the present subject matter.

Further in some embodiments of the present subject matter, the see-through smart device incorporates, an augmented reality interface that overlays the processed information, directly onto the user's field-of-view.

Furthermore, in some embodiments of the present subject matter, the see-through smart device, includes but is not limited to, AR glasses, AR headset, a head-mounted smart device, a smart contact lens, smart-phone and other known see-through smart devices, as known within the art.

Moreover in some embodiments of the present subject matter, the see-through smart device has a number of internal electric/electronic components, that generate the presented processed information, by employing a software application, in order to integrate the received data obtained from the transmitter-receiver unit, by overlaying the presented processed information onto a portion of the body of the biological entity, that is within the field-of-view of the user/medical-practitioner.

Additionally, in some embodiments of the present subject matter, the software application is integrated with machine learning algorithms and generative AI algorithms, to continually improve the detection and visualization accuracy of the foreign entities, over time.

Specifically in some embodiments of the present subject matter, the see-through smart device is equipped with multiple internal electric/electronic components, such as but is not limited to, a memory unit, central processing unit (CPU), graphics processing unit (GPU), power storage unit, modulation units, amplification units, communication units, secondary electronic components, transmitter-receiver units, electromechanical components, camera, as well as, other known essential internal electric/electronic components, as known within the art.

Lastly in some embodiments of the present subject matter, the see-through smart device offers a range of user-friendly features, such as voice-recognition for hands-free operation by the user.

Ultimately in some embodiments of the present subject matter, the see-through smart device is capable of establishing communication with an external database/server, in order to fetch-share real-time information, regarding each of the detected foreign entities, or, even the biological entity.

### Advantage and Utility of the Present Subject Matter

The method and system, for subcutaneous implant visualization, as disclosed within the present subject matter, provides a sophisticated solution, that integrates a see-through smart device having at least one transmitter-receiver unit, in order to deliver detailed, real-time visualizations of foreign entities. This system not only enhances the accuracy of detecting and locating foreign entities, but also supports various medical procedures, through its advanced features and functionalities by providing a clear-differentiation between the biological entity and the foreign entities disposed within it.

Further, the system excels in detecting the exact location/position, shape, size, texture, material, and orientation of the foreign entity. This high level of detail is crucial for effective diagnosis, treatment planning, and other procedural accuracy. By providing comprehensive information regarding the foreign entity's characteristics, the system aids medical practitioners/user in making informed decisions, thereby reducing the likelihood of procedural errors, observational errors, and enhancing the overall quality of care that the patient receives. This precision ensures that, the interventions if any, are accurately targeted, while minimizing risks and improving the overall outcomes.

In one of the embodiment of the present subject matter, the system's detection capability of foreign entities, is not only limited to only human beings, but the system can visualise, detect or localise the presence of any foreign entity, within any of the biological entities, such as but is not limited to, mammal, fish, reptiles, avians, amphibians, plants, insects, molluscs and annelids, thereby enhancing/increasing the scope of the present subject matter, for various research and investigative endeavours, as well as, helps the user in providing a better degree of care, to desired plants and animals.

Additionally, the system is compatible with detection of foreign entities, that are made from diverse materials such as silicon, plastic, metals, composites and their combinations. This versatility allows it to detect, a wide range of foreign entities, regardless of their material and/or composition, making this system a valuable tool in various medical/investigative scenarios.

In one of the embodiments of the present subject matter, the foreign entities are selected from the group consisting of, medical implants, pathological entities, traumatological entities, toxicological entities and other alien objects. In such cases of ailments, the system allows the medical practitioner to visually examine the foreign entity, without cutting open the body of the biological entity, within which that foreign entity resides. This robust and versatile detection capability ensures, that the system can be used across a wide array of medical applications, from detecting common implants like port-a-cath, knee implants, hip implants, cardiac implant, dental implants, joint replacements, etc., to, even other alien objects lodged within the body of the biological entity. This in-turn provides the healthcare professionals with a reliable and comprehensive diagnostic tool.

Furthermore, the system is designed to detect a variety of medical implants, diseases, injuries, etc. This broad detection capability ensures that, the system can be used across numerous medical/investigative disciplines, purposes and procedures, enhancing its utility and effectiveness in various small-scale domains, such as for example, small clinics/labs, medical camps, dental camps, etc.

Furthermore, the system disclosed within the present subject matter, accurately locates the subcutaneous implants, such as for example a port-a-cath, allowing precise needle insertion in a single attempt, reducing patient discomfort and the risk of complications. This precise targeting ensures effective and pain-free drug delivery, by improving the patient experience, while also enhancing the efficiency and safety of such intravenous procedures. Moreover, this precision is vital for procedures, such as needle insertion, where even a slight deviation can lead to serious complications, or, increased patient discomfort.

Further, the see-through device comprises of the transmitter-receiver unit, wherein this transmitter-receiver unit, is very versatile and is integrated into the system in multiple convoluted ways to form a part of a detection ensemble. This ensemble can be embedded within a companion device, that is a detachably-attachable detection device for flexibility, or, be directly embedded within the see-through smart device itself, to streamline the operation. This flexibility of embedding the transmitter-receiver unit in various ways, allows the users to choose from the most convenient and effective method/configuration, for detecting foreign entities based on their various specific scenarios, needs and/or requirements.

Furthermore, the signal processing portion of the transmitter-receiver unit refines the signals, ensuring that only the most relevant and sanitized/normalized data is utilized, which significantly improves the efficiency and effectiveness of the detection process. Additionally, by utilizing advanced signal processing module and combining raw-data from/in multiple frequency ranges, the system is capable of pin-pointing the exact position/location, orientation, texture, material, size and/or shape of a foreign entity within the body of the biological entity, this ensures that the user always has an accurate spatial information, in real-time.

Additionally, the transmitter-receiver unit operates within multiple frequency ranges, such as but is not limited to, Infrared, NIR (Near-Infrared), MRI, MWT, Ultrasound, Ultrasonic, fluoroscopy, PET, endoscopy, arthroscopy, angiography, lymphangiography, mammography, venography, duplex ultrasound, elastography, OCT, EIT, photoacoustic imaging, sonography/radiology, ESI, spectroscopy, ultraviolet, modulated, pulsed, Time-varying, etc. frequencies, in order to be able to detect, visualise and localize, as well as, obtain the orientation, shape, texture, material, size and most importantly the location/position information, of a plethora of different types of foreign entities. Particularly, the near-infrared radiation, has a better penetration depth and enables clear contrast, when interacting with various materials. Furthermore, within an embodiment of the present subject matter, it discloses a subcutaneous visualisation system that employs, near-infrared (NIR) radiation wherein the NIR radiation penetrates tissues, more effectively and interacts very differently with materials, such as silicone. This enhanced interaction improves the contrast and clarity of the detected images, allowing for precise identification and localization of silicone implants. In the present subject matter, the NIR radiation provides a more robust and accurate visualization of specifically subcutaneous/intradermal/visceral implants, ensuring that materials such as, silicone are reliably detected. This advancement allows for more accurate detection and visualization of subcutaneous implants, enhancing the overall effectiveness and efficiency, of the medical procedures.

Further, the transmitter-receiver unit is calibrated to detect foreign entities of different shapes, sizes, textures, materials, orientations and/or materials. This calibration capability ensures that the system maintains high accuracy and effectiveness, across a wide range of foreign entity types, enhancing the system's versatility and utility.

Additionally, the transmitter-receiver unit operates in multiple modes, based on the scenario, such as continuous scan mode and pulse mode, in order to optimize power consumption and detection performance. This enables the system to have an extended battery life, which is Ideal for applications, where power efficiency is critical, or, after the procedure, the pulse mode is activated to monitor the patient periodically to help conserve the battery life, or, in case of remote monitoring systems. Further, the pulse mode is effective for situations where constant monitoring is not necessary, like when detecting implants, here periodic checks are sufficient to ensure that the implant is present in the right location/orientation, thereby enhancing the system's overall functionality and efficiency. This multi-mode capability of the transmitter-receiver unit makes the system versatile and efficient overall, allowing it to be capable of meeting the demands of various applications, from critical medical procedures requiring constant monitoring, to less demanding scenarios where periodic checks would suffice.

In one of the embodiments, the see-through smart device includes an augmented reality interface that overlays the processed information, directly onto the user's field-of-view. This offers numerous advantages, such as but is not limited to, enhancing real-time visualization, improving workflow efficiency, ensuring hands-free operation, increasing accuracy and precision, facilitating better communication, and providing other versatile applications. It represents a significant advancement in integrated patient-care, or, in other words, allows a see-through analysis of biological entities, ultimately contributing to improved healthcare outcomes.

Most importantly, the see-through smart device has various forms, to suit different user preferences and medical environments. These see-through smart devices have a various device types, such as, but is not limited to, AR glasses, AR headsets, head-mounted smart devices, smart contact lenses, smart-phones, etc. Each of these types provides unique advantages in terms of comfort, usability, and functionality, allowing users to select the most appropriate device for their needs.

Further, the see-through device disclosed within the present subject matter has a camera, that enhances the system's ability to document procedures, increases analysis accuracy, and provides a comprehensive visual record of the foreign entity's detection process, as well as, the current status of the biological entity as a video log.

Furthermore, the see-through smart device employs a software application. This application processes, the raw-data received from the transmitter-receiver unit and integrates it after processing it, onto the user's field-of-view, enabling real-time adaptive visualization of the detected foreign entities. Additionally, the system employs machine learning algorithms within the transmitter-receiver unit to continually improve the detection and visualization accuracy of the foreign entities. Over time, the system learns from previous detections, enhancing its overall performance and reliability in various detection contexts, as time passes.

Further, in one of the embodiments of the invention, the subcutaneous visualisation system incorporates voice recognition capabilities wherein the user controls the device without needing to use their hands, maintaining sterility, ensuring real time interaction, user convenience and focus during procedures. This hands-free approach reduces the risk of contamination, minimizes the need for physical interaction with the device, and allows the user to fully concentrate on the task at hand, improving operational efficiency, overall workflow and/or patient safety, compared to other currently known systems, within the art.

Further, the see-through smart device is designed to connect/communicate with external databases/servers, allowing the device to access-transmit real-time information regarding the detected foreign entities. This integration offers several benefits, particularly in the medical field, by providing users with comprehensive and up-to-date details, facilitating better-informed medical decisions, regarding the detected/visualized foreign entity. Specifically, providing detailed information of the detected foreign entities, such as for example: in case when the foreign entity is an implant, in such cases providing the user with the manufacturing details, as well as, version/other data of that implant. Additionally, this also enables the system disclosed within the present subject matter, to send the detection/recorded data/information, as well as, other paraphiliac visualization data/information to the external database/server, which may be used to train other devices in the market, remote-viewing/supervision, or, even to improve the devices manufactured in the future.

Additionally, the accurate detection of silicone implants is crucial for various medical applications, including detection of drug delivery systems, medical/cosmetic implants, and other health monitoring devices. Furthermore, the subcutaneous visualisation system's ability to accurately visualize silicone implants, enhances patient safety and treatment efficacy, providing medical practitioners with the necessary information to make informed decisions and perform procedures with greater precision. In some embodiments, the transmitter-receiver unit, as an ensemble in a companion device, is designed to communicate wirelessly, with respect to, the see-through smart device. This wireless capability enhances the system's ease of use and flexibility, allowing for seamless data transmission and eliminating the need for cumbersome cables.

Further, the transmitter-receiver unit is capable of detecting and distinguishing between, multiple types of foreign entities, simultaneously. This multi-detection capability is crucial, for complex medical scenarios where multiple foreign entities may be present, thus ensuring comprehensive and accurate detection of each specific foreign entity, at all times.

In some embodiments the system includes a user interface, that allows customization of visualization settings, based on user preferences and specific medical/research procedures. This customization capability ensures that the system, is tailored to the needs of the user, enhancing comfort and efficiency, if so desired.

For purposes of summarizing the invention and the advantages achieved over the prior art, certain objects and advantages of the invention have been described above. Of course, it is to be understood that not necessarily all such objects or advantages may be achieved in accordance with any one particular embodiment of the invention. Thus, for example, those skilled in the art will recognize that each embodiment of the invention, may be embodied or carried out, in a manner that achieves or optimizes, one advantage or a group of advantages.

Various components, sub-systems, sub-methods, mechanisms, and/or additional processes, are claimed in the independent/dependent claims, these embodiments may be, combined, or, applied separately, with respect to each other. To the accomplishment of the foregoing and related ends, certain illustrative aspects of the disclosed innovation are described herein, in connection with the following description and the annexed drawings. These aspects are indicative however, of but a few of the various ways in that the principles disclosed herein, as may be employed and are intended to include all such aspects and their equivalents. Other advantages/applications and novel/inventive features will become apparent from the following detailed description when considered in conjunction with the drawings/illustrations provided.

### BRIEF DESCRIPTION OF THE DRAWINGS

The summary above, as well as the following detailed description of illustrative embodiments, is best understood when read in conjunction with the appended drawings/figures. For the purpose of illustrating the present subject matter, exemplary illustrations of the subject matter are depicted within these drawings. However, the present subject matter is not limited to specific methods and instrumentalities disclosed herein.

Moreover, those in the art will understand that the drawings are not to scale/inclusive of all essential components/elements.

Embodiments of the present subject matter will now be described, by way of example only, with reference to the following Drawings/figures, wherein:

FIG. 1 depicts a schematic view of a see-through smart device embodiment 100.

FIG. 2 depicts a schematic view of a see-through smart device embodiment 200.

FIG. 3 depicts a schematic view of a method of implementation of the system, as presented herein, when the foreign entity is a medical implant. FIG. 3A depicts an exemplary scenario of application of the see-through smart device. FIG. 3B depicts a point-of-view of the user, in this exemplary scenario of application of the see-through smart device.

FIG. 4 depicts a schematic view of a method of implementation of the system, as presented herein, when the foreign entity is an alien-entity. FIG. 4A depicts an exemplary scenario of application of the see-through smart device. FIG. 4B depicts a point-of-view of the user, in this exemplary scenario of application of the see-through smart device.

FIG. 5 depicts a schematic view of a method of implementation of the system, as presented herein, when the foreign entity is a blood-clot (for e.g., Thrombophlebitis). FIG. 5A depicts an exemplary scenario of application of the see-through smart device. FIG. 5B depicts a point-of-view of the user, in this exemplary scenario of application of the see-through smart device.

In the accompanying drawings, an underlined number is employed to represent an item over that the underlined number is positioned, or, an item to that the underlined number is adjacent. In other words, an underlined number depicts the whole method/system and is a reference to that embodiment of the method/system, in the specific figure, that it is illustrated/mentioned in. Whereas, a non-underlined number relates to an item identified by a line linking the non-underlined number to the item. When a number is non-underlined and accompanied by an associated arrow, the non-underlined number is used to identify a general item, at that the arrow is pointing to.

### DETAILED DESCRIPTION

The features and advantages of the present subject matter, just as they are stated in the claims, will now be described in detail with reference to the appended drawings, showing several examples of deployment configurations/embodiments for the present invention.

In an overview, most embodiments of the present subject matter relate to subcutaneous visualization system. Some embodiments include providing validated associations of the entity, and the associations may be determined based on at least one or more of the semantics thereof.

The following detailed description illustrates various embodiments of the present subject matter and ways in that they may be implemented. Although some modes of carrying out the present subject matter have been disclosed, those skilled in the art would recognize that other embodiments for carrying out or practicing the present subject matter are also possible. Some disclosed embodiments include one or more systems and methods of, subcutaneous visualization of various foreign entities.

The present subject matter relates to a visualization system, that employs advanced imaging technologies and see-through smart devices, in order to assist the user, to visualise, localise and detect foreign entities disposed within a biological entity, by directly providing real-time processed information to a user. This innovative system is designed to enhance the accuracy and efficiency of medical/research procedures, by clearly differentiating in between, a biological entity (including, all components, parts, portions of the biological entity), and, any kind of foreign entities disposed within the said biological entity. Further, the innovative system enables simple non-invasive method of detecting and visualising, a foreign entity that is inside a biological entity.

The core component of this system is a see-through smart device, that is very similar to, augmented reality (AR) glasses, headsets, smart contact lenses, smart-phones, or, other similar AR display devices. This device essentially, presents the processed information by overlaying it, onto the user's field-of-view, allowing for seamless integration of real-time data onto the user's natural vision. In one of the embodiments of the present subject matter, the see-through smart device is capable of storing historical-data regarding the various foreign entities detected, so far. This feature allows users to access past detection information, for future reference and analysis, aiding in longitudinal studies and improving patient care through comprehensive record-keeping. In another embodiment, the see-through smart device includes an alert mechanism that notifies the user upon successful detection and/or accurate positioning of the foreign entity. This notification may be customized to detect and alert the user regarding any kind of user-desired detection parameters, tailored to detect the specific foreign entity, that the user is interested in.

In one embodiment of the visualization system, it has an ability to provide detailed information regarding the precise location, shape, size, texture, material, and orientation of the foreign entities, as well as, a clear differentiation between foreign entities and the biological entity.

In some embodiments of the visualization system, the biological entities are living beings such as mammals, fish, reptiles, avians, amphibians, plants, insects, molluscs, annelids, etc. The biological entities generally contain several biological components/parts/portions, such as but is not limited to, arteries, veins, bones, muscles, skin, cartilages, ligaments, tendons, blood cells, neurons, immune cells, hair, tissues, organs, phloem, xylem, cambium, pith, pericycle, endodermis, cortex, mesophyll, etc.

In some embodiments of the visualization system, the foreign entities are the entities that are not naturally found inside living beings. These foreign entities are made up of any diverse materials such as, but is not limited to, silicon, plastic, metals, composites, combinations, etc. Further in other embodiments of the present subject matter, the foreign entities are entities, such as but is not limited to, medical/cosmetic implants, pathological entities, traumatological entities, toxicological entities, other alien objects, etc. Furthermore, in some other embodiments of the present subject matter, the system is designed to detect a variety of medical/cosmetic implants, including but is not limited to, port-a-cath, knee implants, hip implants, cardiac implant, dental implants, etc. Further, the system allows a user to insert a syringe-needle into the port-a-cath in a single attempt, by assisting them with the location and orientation of the port-a-cath.

Further, the system includes a transmitter-receiver unit that has a specific-bearing that is dependent upon the type of visualization architecture being implemented. Further, the transmitter-receiver unit is equipped with a signal processing module that filters, amplifies, and converts signals, in order to enhance the detection accuracy of all types of foreign entities. The transmitter-receiver unit, operates within various frequency-ranges/Scanning-technologies, including but is not limited to, Infrared, NIR (Near-Infrared), ultrasound, ultrasonic, MRI, MWT, fluoroscopy, PET, endoscopy, arthroscopy, angiography, lymphangiography, mammography, venography, duplex ultrasound, elastography, OCT, EIT, photoacoustic imaging, sonography/radiology, ESI, spectroscopy, ultraviolet, modulated frequency, pulsed frequency and a Time-varying frequency, ensuring versatile application during various different medical/investigative scenarios. This wide range of operational frequencies ensures that the system is able to effectively detect, various types of foreign entities under different conditions, while providing robust and versatile detection capabilities.

The specific-bearing of the transmitter-receiver unit, is either the see-through smart device itself, or, an additional companion device. Further, in some embodiments of the present subject matter, this companion device is detachably attached to the see-through smart device. Furthermore, the companion device is capable of maintaining communication with the see-through smart device, even when the companion device is physically detached from the see-through smart device. Moreover, in other embodiments of the present subject matter, the see-through smart device, has a plurality of companion devices, that are detachably attached to the see-through smart device, at various different predefined locations on the see-through smart device, in order to gain the most optimal perspective. In yet other embodiments, the companion device is a separate clip-on device, or, a handheld/portable device. Importantly, in some of the embodiments of the present subject matter, the see-through smart device, communicates with a plurality of companion devices, that enable the see-through smart device, to obtain either, unprocessed raw-data, or, processed information, with respect to the detected/identified entities (biological, or, foreign). This enables the see-through smart device, to provide an added depth/resolution to the processed information, being presented to the user, when utilising some of the heavier companion devices, that operate by utilising several medically-safe frequencies/scanning-techniques, such as, but not limited to, Infrared, Near-Infrared, X-rays/CT (computed tomography), MRI (magnetic resonance imaging), MWT (microwave tomography), Ultrasound/Ultrasonic, fluoroscopy, PET (positron emission tomography), endoscopy, arthroscopy, angiography, lymphangiography, mammography, venography, duplex ultrasound, elastography, OCT (optical coherence tomography), EIT (electric impedance tomography), photoacoustic imaging, sonography/radiology, ESI (electrical source imaging), spectroscopy techniques (such as, infrared Spectroscopy, Raman Spectroscopy, Fluorescence Spectroscopy, and most importantly Surface-enhanced Raman Spectroscopy techniques), ultraviolet radiation/frequencies, and other such frequencies/scanning-techniques used within the medical field, as known within the art. In yet another embodiment, the various transmitter-receiver unit/s (lamps, LEDs, acoustic generators, sensors, arrays, transmitters, receivers, emitters, detectors, signal processing modules, etc.) are embedded within the see-through smart device, in this case each of the transmitter-receiver unit, will transmit/emit a signal, this signal/wave/photon will travel through the air-medium (space between the user's head and a biological entity) to reach the biological entity. After that it penetrates the dermal layers of the biological entity, to reach deep within the body of the biological entity. Here, the signal/wave/photon gets reflected back from the foreign entities and some of these reflected signals/waves/photons travel back to the receivers/array/sensors/detectors. These signals/waves/photons that are received back, by the transmitter-receiver unit will then be processed, in order to generate images of the biological entity, as well as, the foreign entities, which is constructed based primarily on this type of raw-data. This information is then projected onto the screen of the see-through smart device, clearly differentiating between both, the biological entity and the various foreign entities disposed within the biological entity. Lastly, in some embodiments of the present subject matter, the companion device, is detached from the see-through device during detection/investigation process, to then be temporarily coupled to a magnification/enhancement equipment, which increases the strength of the companion device, in terms of visualization capabilities.

Specifically, the transmitter-receiver unit is calibrated to detect foreign entities with varying sizes, shapes, texture, material, orientations and/or their location/position. Further, in some embodiments of the present subject matter, the transmitter-receiver unit operates in multiple modes based on the scenario, including but is not limited to, continuous scan mode, pulse mode, etc., in order to optimize the consumption of power and detection performance. Further, in continuous scan mode, the transmitter-receiver unit constantly emits signals/photons/waves and receives/detects/senses for responses without any interruption, leading to a higher performance rating, that is ideal for critical applications, such as during surgical procedures, detailed research-analysis endeavours, or, even in high-security environments. Furthermore, in pulse mode, the unit emits signals/photons/waves in short-intermittent bursts and receives/detects/senses for responses during the intervals between these pulses. This flexibility allows users to optimize power consumption and detection performance based on their specific needs, thereby enhancing the system's overall functionality and efficiency. In one of the embodiments of the present subject matter, the transmitter-receiver unit includes an auto-calibration feature to automatically adjust its settings to maintain optimal performance. This auto-calibration process does not require manual intervention, making the system more user-friendly and efficient.

To further improve the system's functionality, the see-through smart device also consists of a camera, in some embodiments of the present subject matter.

Further in some embodiments of the present subject matter, the see-through smart device incorporates, an augmented reality interface that overlays the processed information directly onto the user's field-of-view. This AR capability allows the users, medical-practitioners, researchers, analysts, etc., to visualize various types of foreign entities, their specific location and other paraphernaliac details, without diverting their attention from the biological entity or patient, facilitating more precise and intuitive medical/research-analysis procedures, in real-time.

Furthermore, in some embodiments of the present subject matter, the see-through smart device, includes but is not limited to, AR glasses, AR headset, a head-mounted smart device, a smart contact lens, smart-phones, and other known see-through smart devices, as known within the art.

Moreover in some embodiments of the present subject matter, the see-through smart device has a number of internal electric/electronic components, that generate the presented processed information, by employing a software application, in order to integrate the entirety of the received raw-data obtained from the transmitter-receiver unit, by overlaying this presented processed information onto a portion of the body of the biological entity, that is within the field-of-view of the user/medical-practitioner.

Additionally, in some embodiments of the present subject matter, the software application is integrated with machine learning algorithms and generative AI algorithms, to continually improve the detection and visualization accuracy of the foreign entities, over time. The software application processes the data/information (signals/waves/photons) received/detected by the transmitter-receiver unit and integrates them after processing, onto the user's field-of-view, enabling real-time adaptive visualization of all the detected foreign entities. In one of the embodiments, these detected foreign entities are any kind of unmodified medical/cosmetic implants.

Specifically in some embodiments of the present subject matter, the see-through smart device is equipped with multiple internal electric/electronic components, such as but is not limited to, a memory units, central processing units (CPU), graphics processing units (GPU), power storage units, modulation units, amplification units, communication units, secondary electronic components, transmitter-receiver units, electromechanical components, cameras, as well as, other known essential internal electric/electronic components, as known within the art. These components work together, in order to ensure that, the device operates efficiently and effectively under the commands received from the software application.

Lastly in some embodiments of the present subject matter, the see-through smart device offers a range of user-friendly features, such as voice-recognition for hands-free operation by the user. This feature is particularly useful when the system is being utilised within a sterile environment, where manual control might be impractical or undesirable, allowing users to control the system via, customizable user-defined voice commands.

Ultimately in some embodiments of the present subject matter, the see-through smart device is capable of establishing communication with an external database/server, in order to fetch-share real-time information, regarding, each of the detected foreign entities, or, even the biological entity. Further, these external databases/servers contain extensive information, including but is not limited to, for example, in case the foreign entity is any type of subcutaneous/intradermal/visceral implants, then the servers/databases will obtain, the implant data from manufacturers, regulatory bodies, historical data regarding that specific implant, or any other useful information pertinent to that implant, as is known within the field in such a scenario. In one of the embodiments, the see-through smart device further includes, a remote-control feature, allowing another user/senior medical-practitioner to be able to guide the primary user, during complex procedures. In yet another embodiment, the see-through smart device is designed to be compatible with telemedicine platforms, allowing remote medical/research experts to be able to assist and guide the user in real-time, as well as, allow the remote medical/research experts to remotely examine the interior portions of the biological entity and especially the disposed foreign entities within this biological entity.

### Definitions of Terms Used

In the case, where there are two or more definitions of a term that is used and/or accepted within the known art, the definitions of the terms as used herein, is/are intended to include all such meanings unless explicitly stated to the contrary.

For purposes of the detailed description of the preferred embodiments, the definitions of most of the essential terms, that are utilised and enforced, throughout the present subject matter including the claims, are provided as follows:

Throughout the present subject matter, the term the “see-through smart device” refers to, an electronic device that is worn/held on by the user, that primarily enables a visual presentation of subcutaneous/intradermal/visceral foreign entities, that are not usually visible, to the naked-eyes of the user. The see-through smart device takes various forms, in order to suit the different user preferences and medical/research environments. The see-through smart device is any form of device, such as but is not limited to, augmented reality (AR) glasses, AR headset, head-mounted smart device, smart contact lens, or, any other form of see-through smart device, as known within the field. In certain other embodiments of the present subject matter, alternatively the see-through device, is a smart-phone, wherein the screen/display of the smart phone, overlays and presents a subcutaneous foreign entity, over the real-time image/video feed, of the biological entity as captured by the rear-camera of the smart phone. Each type of the see-through smart device, provides unique advantages in terms of comfort, usability, and functionality, allowing the users to select the most appropriate device/mode, suited/tailored for their specific needs. The see-through smart device presents the processed information, by overlaying the processed information onto the user's field-of-view, allowing for seamless integration of this real-time data/information, onto the user's natural vision, or, their specific field-of-view. Primarily, the visualization system depends extensively, on the capability of the see-through smart device, in order to be able to provide the user with detailed information, regarding the precise location/position, shape, structure, texture, material, and/or orientation of the visualized/detected foreign entities, and especially, in displaying a clear differentiation to the user, in-between these said foreign entities and the biological entity that contains these foreign entities. Essentially, the see-through device, employs an augmented reality interface, that overlays the processed information directly onto the user's field-of-view. Hence, the see-through smart device comprises several internal electronic/electrical components, to enable the processes, as described above. These components are responsible, for generating a processed information, by employing a software application, in order to integrate a raw-data obtained from a transmitter-receiver unit, that undergoes processing to form a processed information, that is then overlaid onto a portion of the body of the biological entity, that is within the field-of-view of the user/medical-practitioner. It is understood by anyone skilled in the art, that various display technologies may be employed, by the see-through smart device as one of the internal electronic/electrical components, disclosed within the present subject matter, in order to be able to present, the above described processed information, that is overlaid onto a user's field-of-view. These display technologies are, such as but is not limited to, Transparent OLED (T-OLED), Micro-LED, Holographic Waveguides, Liquid Crystal-based Display (more specifically, referring to partially transparent LCD), Projection-based Display, Electrochromic Display, Quantum Dot Display (more specifically, referring to Nanocrystal-based display), and other similar display technologies that enable see-through capability, that are selected based on the specific application-area, desired image-quality, power constraints and several other considerations like, raw-materials utilised, costs, sustainability, manufacturability, serviceability, reliability, safety and other unforeseen challenges. These display technologies employed by the see-through smart device, make up a core portion of the augmented reality interface of the see-through smart device, that is responsible for providing a processed information to the user. These display technologies employed by the see-through smart device, additionally offer adjustable transparency levels, optimizing visualization settings based on the ambient lighting conditions at that moment, and/or several other features such as, for e.g., different type of alert protocols upon detection of different kinds of foreign entities, using contrasted colours for highlighting detected foreign entities, providing clear digitally-drawn outline for the detected foreign entities, employing a visual eye-catching mechanism (such as, for e.g. a blinking region in the field-of-view, or, other visual tags or markers, etc.) to ensure that the user notices/acknowledges (either, by blinking/winking back at a blinking region on the field-of-view, or, by gazing in the direction of the said blinking region for a few seconds, whichever is preferable to the user) each of the detected foreign entities in their field-of view. This adaptability ensures that the overlaid processed information always remains visible, legible, at-par with ambient brightness and well-focused on to the user's field-of-view, regardless of the lighting conditions, ensuring that the user never misses any intended foreign entities even by mistake, in-turn enhancing the overall user experience. This adaptability of being able to, adjust the change in transparency, means the user gains a degree of control with regards to, how much they see through the said device. For example, in bright sunlight, the device becomes less transparent, to enhance the images and/or text displayed on it, so that it is easily seen by the user. Alternately, in darker environments, the device becomes more transparent, so that the user is able to see through it better. This adjustable transparency helps ensure that, the user always enjoys a clear and optimal view of the processed information, regardless of the lighting conditions. In one of the embodiments, the see-through smart device, employs a plurality of different types of memory units as one of the internal electronic/electrical components, that are capable of storing historical data of the detected foreign entities, of past. This feature allows a user, to access past detection information for future reference and analysis, aiding in longitudinal studies, as well as, improving patient care through comprehensive record-keeping in visual, auditory and/or textual form. In another embodiment, the see-through smart device includes, an alert mechanism that notifies the user upon successful detection and accurate positioning of a foreign entity. This immediate feedback ensures that, users are made aware of the foreign entity instantaneously, aiding in quick and informed decision-making.

Further, in many of the embodiments of the present subject matter, the see-through device includes a plurality of internal electric/electronic components, such as but is not limited to, memory units, central processing units, graphics processing units, power storage units, modulation units, amplification units, communication units, secondary electronic components, transmitter-receiver units, electromechanical components, camera and other necessary components, in order to, be able to perform all the tasks listed herein, as well as to, enable all of the features of the proposed system and method, as described within this present subject matter. However, it should be noted that, it is not necessary that, all the above-mentioned components must be present within the see-through smart device, in order for the method and system described herein to be implemented successfully, as would be easily understood and very apparent, to a person skilled within the art. Furthermore, in many of the embodiments of the present subject matter, the see-through device has one or more companion device/s, these companion devices include a plethora of internal electrical/electronic components, same as described-above for the see-through smart device. The primary purpose of this companion device, is to assist the see-through smart device with their ability to, identify, predict, scan, detect, identify, and process the various characteristics, such as but is not limited to, the location/position, shape, size, texture, material, orientations and/or materials, of a variety of subcutaneous foreign entities, that are disposed within a biological entity. While the secondary purpose, of this companion device is to relay a processed information, or, in other instances just the obtained raw-data (dependent on the characteristics of the utilised type of companion device architecture and other related capabilities), to either a processing unit, or, directly to the display technology that is being utilised, for presenting the processed information to the user. This relay of processed information/raw-data, between the see-through smart device and the companion device is conducted either, wirelessly, or, via a separate wired-connection, as per the particular specific requirement of the user/administration, as is known within the art. Moreover, in many of the embodiments of the present subject matter, the see-through smart device includes voice recognition capabilities integrated with generative AI algorithms and models, such as but not limited to, speech detection, sanitization/normalization of received audio input, language detection, pronunciation/accent adjustments based on location of user, populate the user-query with intelligently predicted data to rectify any kind of missing words/phrases/information, etc., in order to enable hands-free operations by the user, while also enabling interactive/informative conversations with the data/information, that is being presented to the user, in real-time. This feature is particularly useful in environments that require extremely sterile conditions, where manual control might be impractical or undesirable, allowing the users/medical-practitioners to control the system via, only voice commands. Further this feature, allows the user to go through all the data/information available at the time in an intelligible way, with the assistance provided by the generative AI chat tools, in real-time.

In one of the embodiments, the see-through smart device employs, a plurality of different kinds of communication units, within the plethora of internal electronic/electrical components that the device has, these communication units are configured to connect with a plurality of external databases/servers over the internet, or, through private secure-networks, allowing the device to access, as well as, provide real-time information both-ways, regarding the detected foreign entity and the biological entity, both. The communication units operate on a wide-variety of wired/wireless communication architectures, such as but is not limited to, LAN, WAN, Ethernet, HDMI, Bus-Cables, USB, Type-C, Wi-Fi, cellular networks, Li-Fi, Lo-Ra, Bluetooth, NFC, Multiplexed, Infrared, IOT, or, in some instances the device may connect locally with a smart-phone to establish communications with an external database/server through the smart-phone, and these communication architectures are selected based on the specific user/administrator requirements. These external databases/servers contain extensive information, including but is not limited to, the various types of subcutaneous implants, data from manufacturers for implants, guidelines from medical regulatory bodies (especially the procedures and precautions, with respect to the scenario), EHR/EMR (electronic health/medical record), historical-data regarding the detected foreign entity (if any available, for e.g., past data relating to the foreign entity, all/any medical procedures related to the foreign entity, bullet/shrapnel trajectories that were detected in the past, subcutaneous visual patterns/signs as exhibited by any detected pathogen in the past, etc.), or, any other information that is made available, to the database/server, a sperate secure-server, and/or, the see-through device itself. This information is made available to the see-through smart device, by either, the user/institute/organization, or, the biological entity (in case, the biological entity is a human). In one embodiment of the present subject matter, the information about the intradermal implants, including but is not limited to, the implant's manufacturer, model number, material composition, intended use, expiration date, any specific handling or care instructions, other important implant specifications that are made available, as the external database/server constantly updates itself with this type of data/information from various available sources, as described above. Essentially, the historical data regarding the implant includes, but is not limited to, previous check-ups, electronic health/medical record, any other reported issues, patient/implant maintenance records, or, any other implant-based historical data, as known within the field/art. The implant specification information includes, but is not limited to, any of the implant's dimensions, materials, special-features, requirements (for ensuring its compatibility, with other medical devices or treatments), and other similar specifications, as known within the art. In some embodiments, the see-through smart device is capable of communicating with other medical devices and systems, facilitating integrated patient care, in case it is so desired by the user, administrator, institute, organization, etc. that is employing the system and method, as disclosed within the present subject matter. This interoperability enhances the system's utility in a comprehensive healthcare environment, enabling highly coordinated and efficient medical procedures. For e.g., the see-through smart device is capable of, acquiring past, infrared, NIR (near-infrared), MRI, MWT, Ultrasound, Ultrasonic, fluoroscopy, PET, endoscopy, arthroscopy, angiography, lymphangiography, mammography, venography, duplex ultrasound, elastography, OCT, EIT, photoacoustic imaging, sonography/radiology, ESI, Spectroscopy, and other similar medical imaging related raw-data/information from a central-hub of smart connected devices, that are available on a medical server of a hospital (for instance), in order to, primarily improve the resolution of information that the device is obtaining from the transmitter-receiver units employed by the device, and, secondarily processing the obtained data, while populating gaps within the detected information, by utilising this acquired past data wherever appropriate. This enhances the capability of the device to accurately, predict, scan, detect, and identify, various characteristics, such as but is not limited to, the location/position, shape, size, texture, material, orientations and/or materials, of a variety of subcutaneous foreign entities, that are disposed within a biological entity. In another one of the embodiments, the see-through smart device further includes, a remote-control feature, allowing another user/administrator to guide the primary user during any type of complex procedures/investigations, this capability assists multiple users to collaborate, to essentially enable them in making collective/informed decisions in real-time. This remote assistance capability is particularly valuable in training scenarios, or, when expert guidance is required, enhancing procedural accuracy and outcomes. In another embodiment, the system includes a user interface that allows customization of detection/identification/visualization settings, based on several factors, such as but is not limited to, user/administrator preferences, specific medical procedures, requirements of a/an investigative-project/research-experiment, etc. This customization capability ensures that users are capable of tailoring the system to their specific requirements, enhancing comfort, ease-of-operation and overall-efficiency. In yet another embodiment, the see-through smart device is designed to be compatible with telemedicine platforms, allowing remote-experts to assist and guide the user in real-time. This telemedicine integration expands the system's utility, enabling remote consultations and enhancing access to advanced insightful/expert medical advice on demand, particularly this feature has not been available in the past. Lastly, it must be noted that, in any of the following embodiments, the see-through smart device employs more than one companion device, that is/are detachably attached, or, in other scenarios is/are completely physically separate device/s, that primarily carry their own transmitter-receiver units, in order to detect/identify/visualize the various foreign entities and relay this obtained raw-data, or, processed information, to the see-through smart device.

Throughout the present subject matter, the term “processed information” refers to, raw-data that has undergone digital manipulation through various computational techniques in order to, enhance its usefulness, resolution and clarity. This raw-data that has been obtained by the various types of transmitter-receiver units, that are directly/indirectly employed by the see-through smart device, in order to obtain various types of raw detection data/information, regarding the subcutaneous/visceral layers of the biological entity of interest and, especially the various kinds of foreign entities disposed therein, as per the interest of the user/administrator-of-device. The digital manipulation of this raw-data includes sorting, filtering, aggregation, analysis, and other forms of data processing techniques, as known within the art. The goal here, is to transform the obtained raw-data, into a more structured and meaningful format, so that it is easily interpreted and utilized for decision-making, reporting, or further analysis. In one of the embodiments, the processed information also utilises, historical information of the patient consisting of past health related aspects, such as but is not limited to, for e.g., past cancerous tumour scans, past muscle-tear MRI scans, past X-ray information, etc., in order to collate the obtained raw-data with this historical information, to ultimately provide a higher resolution of processed information to the user, while minimizing the processing power required to achieve this quality, precision and accuracy. It must be noted that, in any of the embodiments of the present subject matter, the processed information includes, but is not limited to the, precise location/position, shape, texture, material, structure and/or orientation of the various types of foreign entities, with respect to the subcutaneous/visceral portions of the biological entity, that is/are present in front of the user's gaze. The processed information is generated by the various internal electric/electronic components of the see-through smart device, after filtering, amplifying, modulating and converting the obtained signals/raw-data from the various transmitter-receiver units of the see-through smart device, in order to eventually coalesce the entirety of the available information, raw-data and other acquired data/information, to finally provide the processed information as an output. This process of generating the processed information is accomplished with the help of several different complex algorithms, programs and other computer models, which encompasses the software application, that is employed by the see-through device, in order to enhance the accuracy and reliability of the signals sent-received by the plurality of transmitter-receiver units, as well as, mainly accomplish the generation of the final processed information by utilising the various internal electric/electronic components of the see-through device, as described above. By refining/controlling the various transmitted/received signals, the software application ensures that only the most relevant and precisely obtained data/information is utilised, for generating the processed information, in turn significantly improving the efficiency and effectiveness of the detection/identification process of any specific foreign entity, by the transmitter-receiver unit. Throughout the present subject matter, the term “filtering” refers to the process of removing unwanted/non-pertinent components/features from a signal obtained, by the receiver portion of the transmitter-receiver unit, ensuring only a pertinent portion of this data is processed. These components/features are of various types such as, but not limited to, noise, power line interferences, random signal interferences, echo of same signal and other irrelevant signals that are too weak, or, provide unclear resolution of detection. These filters are utilised primarily to isolate and relay only certain aspects of the obtained signals/raw-data, while suppressing most others. These filters are of various types, including, but not limited to, Low-Pass Filters, High-Pass Filters, Band-Pass Filters, Band-Stop Filters (Notch Filters) and any other similar signal filters, as known within the art. Throughout the present subject matter, the term “amplifying” refers to the process of increasing the power or amplitude of an obtained-filtered signal/raw-data. Essentially, the amplification process improves/boosts the strength of the filtered signal, using several known programmes/algorithms that are based on, such as but not limited to, complex-mathematics, calculus, statistical, probabilistic, computational, and/or other similar amplification software models as known within the art, in order to enhance the quality of the obtained signals by increasing their amplitude relatively more than, all the other irrelevant, unnecessary, or, unwanted noise, if any remains unfiltered after the previous filtering step. A dedicated amplifier unit is utilised in some of the embodiments, that is for example, including but is not limited to, Audio Power Amplifier, RF Power Amplifier, DC Power Amplifier and any other amplifier as known within the art. Throughout the present subject matter, the term “modulate” refers to the process of modulating these filtered-amplified digital signals into useful information, that provides the software application, various kinds of data/information regarding both the biological entity and the various kinds of foreign entities that are disposed subcutaneously within this biological entity. This modulation process essentially enhances the reliability of signal transmission, by allowing signals to be transmitted over long distances, through various media, and especially within different environments, without incurring any significant loss of data/information. This also enables multiple signals to share the same channel (multiplexing), increasing the efficiency/resolution of the detected/identified signals. Throughout the present subject matter, the term “convert” refers to the process of converting these filtered-amplified-modulated digital signals into presentable processed information, that is provided by the software application along with the help of an augmented reality interface, in the form of various kinds of visual/representation information to the user, specifically pertaining to both the biological entity and the various kinds of foreign entities, that are disposed subcutaneously within this biological entity. Throughout the present subject matter, the term “detection” or “identification” refers to the process of detecting within both, these filtered-amplified-modulated-converted digital signals, as well as, more specifically the originally obtained raw-data, in order to cumulatively detect/generate a clear-differentiation between the biological entity within the user's field-of-view, and the foreign entities within this biological entity. These clear-differentiation may then be presented as, for example, but not limited to, a highlight, a blink, or, an alert to the user, by supplementing the displayed real-time processed information, especially including this crucial clear-differentiation. Further, the processed information, in any of the embodiments, essentially encompasses all the displayed information to the user, that also contains any/all the pertinent information that the user desires, including but not limited to, the aforementioned processed information, historical records, patient information, procedures, precautions, regulations, etc., as per the requirements of the user/administrator-of-device. To summarise, within any of the embodiments of the present subject matter, the various internal electric/electronic components of the see-through smart device (also including, any/all companion devices that are in communication with the see-through smart device), that generate the processed information by employing a software application, after integrating the various raw-data/signals that are obtained/acquired from the plurality of employed transmitter-receiver units by the device at that time, in order to then generate processed information by processing these raw-data/signals, similarly to the methods described above, or, other methods, as known within the art, to then be able to, display this processed information, onto a portion of the body of the biological entity, that is within the field-of-view of the user of the see-through smart-device, in real-time. It must be noted that, the accuracy of the real-time data is very much dependent, on the stability of the biological entity, as any dynamic motion/moment by the biological entity may distort the processed image significantly, hence the user must always ensure that, the biological entity is as stable and static, as is possible under the circumstances, for experiencing the maximum efficiency and best quality of the system and method, as described within the present subject matter. Furthermore, this processed information is directly overlaid onto the user's field-of-view, by utilising an augmented reality interface of the see-through smart device.

Throughout the present subject matter, the term “clear-differentiation” refers to the visually distinct separation that is presented between the perceived location/position, shape, size, material, orientation and/or texture of different foreign entities, with respect to a biological entity and its various subcutaneous/visceral layers/contents/components, within the obtained raw-data, or, processed information. This clear-differentiation is achieved owing to the fact that, the refractive index of different objects is different, or here, more importantly the various properties/characteristics, such as but is not limited to, materials, textures, shapes/sizes (especially, when dealing in below micro-meter ranges), chemical compositions, polarizability, dispersibility-of-light, specific wavelength of light-beams that each object absorbs/reflects and many other such characteristics/properties that affect the refractive index of any object, etc., are significantly different in the case when considering the various foreign entities of interest, and comparing them with respect to the biological entity (including, all of its subcutaneous/visceral contents/components). The term refractive index is defined scientifically, as the refractive index (or refraction index) of any object/entity, which is a dimensionless number, that indicates the light bending ability of that medium. The refractive index determines how much the path of light is bent, or refracted, when entering a material. This is described by Snell's law of refraction, n1 sin θ1=n2 sin θ2, where θ1 and θ2 are the angle of incidence and angle of refraction, respectively, of a ray crossing the interface between two media with refractive indices n1 and n2. The refractive indices also determine the amount of light that is reflected when reaching the interface, as well as the critical angle for total internal reflection, their intensity (as provided by, Fresnel equations and Brewster's angle). These considerations and other known methods within the field, are employed and taken into account by the see-through smart device, or more importantly the software application that this device employs, in order to detect/identify and present this clear-differentiation, between each of the foreign entities that are disposed within a biological entity, with respect to, the biological entity, as well as the various subcutaneous/visceral contents/components/parts of that biological entity. Further, this clear-differentiation is provided/presented to the user, either visually, or, auditorily. This presented visual clear-differentiation, in some of the embodiments involves showing/presenting to the user, all of the various crucial/critical portions (subcutaneous/visceral/entrails regions) of the biological entity, that are near-by/close-to the foreign entities, that are being detected/identified by the user, in the scenario. This visual information is provided to the user, so the user may try to avoid coming into contact with any of these critical regions, during the procedure/investigation. While, the presented visual clear-differentiation provided to the user, also includes, but is not limited to highlighting of corners/edges of the foreign entities, depiction of texture/material changes over the various portions of each of the foreign entity, size of each of the foreign-entity including the depth information (with the help of 3D-modeling algorithms), possible trajectories of accessing the foreign entity while avoiding any/all critical portions of the biological entity, comparison information between a previous visual scan with a recent visual scan to visualize/calculate the rate of a foreign entities progression through the biological entity (particularly, in case of pathological/toxicological entities), etc. and providing these as clutter-free background-contrasted visual items, such as, but not limited to, symbols, markers, flags, highlighting, shadows, animation, pointers, colour/shape varying symbols/icons (to alert regarding vital signs of a patient, such as but not limited to, heartrate, blood pressure, oxygen concentration, etc.), their combinations, as well as other known visual attention capturing tools within the filed, are populated all over the field-of-view of the user. These clutter-free background-contrasted visual items may provide the necessary information that they are holding upon interaction by the user, to elaborate regarding that visual item, by either gazing, winking, blinking, using voice-commands, etc. in order to interact, as well as, toggle each item whether the information is required at that time or not. Similarly, the presented auditory clear-differentiation, may be audio alerts customized by the user, with respect to various possible occurrences during the course of a subcutaneous visual examination/identification/detection, with respect to at least all the generic scenarios that are possible. For e.g. the see-through smart device provides, for example, but is not limited to, a varying-interval/pitch-varying sound with respect to, where the hands of the user are in the field-of-view, with respect to the various critical portions of the biological entity, a medical implant, to pinpoint the location of a foreign entity (very similar beeping made by a metal detector, used on a sandy bay, when it identifies a metal object for instance), patient's heartrate/blood-pressure/respiratory rate safety-limit breach, etc., as known within the field. Furthermore, in some of the embodiments of the present subject matter, the clear-differentiation information, is acquired from a plethora of past scanning data and/or with the assistance of a plurality of real-time transmitter-receiver units of the system.

Throughout the present subject matter, the term “biological entity” or “patient” refers to, any life-forms, that exists independently, as well as, carries out all of the essential functions of life, or, simply understood as any entity/thing/being, that is considered living based on a strictly medical/biological point-of-view. These biological entities, are typically composed of multiple/plurality of a variety of cells, tissues, organs, and complex organic/biological systems and sub-systems, that all work together to maintain homeostasis and respond to any kind of an environmental stimuli. These biological entities generally contain several biological components/content/parts/portions, such as but is not limited to, arteries, veins, bones, muscles, skin, cartilages, ligaments, tendons, blood cells, neurons, immune cells, hair, tissues, organs, phloem, xylem, cambium, pith, pericycle, endodermis, cortex, mesophyll, and any other such biological components/contents as known within the art. These biological entities are living beings, such as but is not limited to, mammals, fish, reptiles, avians, amphibians, plants, insects, molluscs, annelids, and any other living beings, as known within the art. Throughout the present subject matter, the term “mammals” refers to any type of vertebrate animals, such as but not limited to, human beings, lions, tigers, elephants, horses, goats, cats, bats, dolphins, whale, or, many other known mammals within the art. Throughout the present subject matter, the term “fish” refers to any aquatic, anamniotic, gill-bearing vertebrate life-forms with swimming fins and a hard skull, such as but not limited to, salmon, tuna, clownfish, sharks, jellyfishes, octopi, or many other types of fish, as known within the art. Throughout the present subject matter, the term “reptiles” refers to a group of animals with an ectothermic (‘cold-blooded’) metabolism and amniotic development, such as but not limited to, snakes, lizards, crocodiles, turtles, or any other reptile known as within the art. Throughout the present subject matter, the term “avians” refers to egg-laying, winged, vertebrate animals that constitute the class avian, such as but not limited to, sparrows, pigeons, flacons, ducks, pelicans or any other avians, as known within the art. Throughout the present subject matter, the term “amphibians” refers to ectothermic, anamniotic, four-limbed vertebrate animals that constitute the class Amphibia, such as but not limited to, frogs, salamanders, newts or any other amphibians, as known within the art. Throughout the present subject matter, the term “insects” refers to hexapod invertebrates of the class insecta, that form the largest group within the arthropod phylum, characteristically having a chitinous exoskeleton, a three-part body (head, thorax and abdomen), three pairs of jointed legs, compound eyes, and a pair of antennae, such as but not limited to, butterflies, ants, and beetles, or any other insects, as known within the art. Throughout the present subject matter, the term “molluscs” refers to a large phylum of invertebrate animals that have a soft, unsegmented body, and most have a calcareous shell, such as but not limited to, snails, clams, and squids, or any other molluscs, as known within the art. Throughout the present subject matter, the term “annelids” refers to a phylum of invertebrate worms characterized by their segmented bodies, that characteristically have a coelom, that is a fluid-filled body cavity that is lined with tissue derived from mesoderm, such as but not limited to, earthworms, leeches, and polychaete worms, or any other annelids as known within the art. Essentially, the biological entity is any life-form, that is under observation by the user of the system/method of the present subject matter. This life-form is in many cases a patient in a medical setting, however, the life-form may also be a wild entity that is being researched/investigated by a researcher/scientist by employing this system/method, owing to the wide-variety of suitable applications that are made possible by the present subject matter, as described herein. In yet other embodiments, the biological entity could be any type of meat, or, dead animal, especially for human consumption, where prospecting/investigative user/journalist, utilises the see-through smart device, as described within the subject matter of this presented system and method, in order to detect/identify any foreign entities, such as for example, plastic, diseases, injuries, etc., as per the user's desire, within this meat/dead-animal.

Throughout the present subject matter, the term “foreign entities” or “foreign entity” mainly refers to, the entities that are not supposed to be naturally found inside the living beings, or, in other words are foreign to any particular life-form/biological entity. Further, the foreign entity/entities as described throughout the present subject matter, relate mostly to an unmodified passive foreign entity, meaning that the foreign entities do not have any kind of inherent modifications of any kind that enable them to exhibit/emit any kind of radiation, frequency, and/or signal, on their own, or reflects back any kind of specifically tailored detection signal, when the foreign entity is pinged utilising a pre-defined signal. Hence, the foreign entities are any components/objects, such as, but not limited to, implants, silicone objects, metal objects, glass objects, plastic objects and any other foreign entities, as understood by a person skilled in the art, that are essentially not modified in any way, in order to assist in detection/identification of these foreign entities. Further, these foreign entities are any kind of medical/cosmetic implants, pathological entities, traumatological entities, toxicological entities and/or other alien objects. Further, the “implants”, “medical implants” or “cosmetic implants” refer to the devices or artificial-tissues that are generally surgically placed inside a biological entity. These medical/cosmetic implants can either be temporary or permanent, and are used to replace or support damaged or missing portions of the specific biological entities, as per their specific needs. The medical/cosmetic implants are objects, such as but not limited to, pacemakers, artificial joints, knee-implants, hip-implants, cardiac implant, dental-implants, plates, stents, breast implants or any other implanted objects, as are known within the art. Further, more specifically intradermal implants are a type of medical implants, that are utilised for a variety of medical applications, such as but not limited to, controlled drug delivery, continuous monitoring of physiological parameters (e.g., glucose levels in diabetes patients), or providing localized therapeutic effects. These intradermal implants are designed to be minimally invasive and remain within the skin/body of a biological entity for extended periods of time, offering a sustained and controlled release of medication or continuous monitoring without the need for frequent replacements or external devices. Further, the system/method of the present subject matter, detects these intradermal implants, that are made from various diverse materials, such as, but not limited to, silicon, plastic, metals, composites, their combinations, or any other suitable materials, as known within the art. Additionally, the system/method is designed/calibrated to be able to detect/identify a variety of these unmodified intradermal implants, such as but is not limited to, port-a-cath, knee implants, hip implants, cardiac implant, dental implants and any other similar intradermal implants as are known within the art. Further, the “pathological entities” refers mainly to the disease-causing agents or conditions within the body, which are also considered as pseudo-foreign entities in some embodiments, mainly due to the reason that, these albeit are biological entities, are single-celled organisms which primarily are causing harm/malice to the biological entity of interest. These pathological entities are mostly responsible for causing a range of health issues, from infections to chronic diseases. These pathological entities are, such as but not limited to, bacteria, viruses, fungi, parasites, prions, as well as, other abnormal growths such as cancerous-tumours, cysts, blood-clots, or any other such agents, as are known within the art. The system/method of the present subject matter, utilises the various differences in the refractive index of these agents, in order to differentiate them from the portions of the biological entity, by providing a clear picture regarding a rate-of-spread (between consecutive scans/detections) and the affected-areas by these agents, within the biological entity without using an intrusive methods, as in the past. Further, the “traumatological entities” refers to injuries or trauma to the body of the biological entity. The Traumatological entities are injuries or trauma, including but not limited to, depth of the cut, cell lysis area, tissue damage, internal haemorrhaging, muscle tear/damage, bone crack/damage, lacerations, contusions, internal injuries, or any other injuries/trauma, as are known within the art. This provides, insights with respect to several aspects relating to the healing characteristics of these injuries/trauma to a user, without employing extensive intrusive methods of investigation. Further, the “toxicological entities” refers to the toxic substances that cause harm to the body, leading to poisoning, organ damage, or other health complications. These toxicological entities are toxic substances, including but not limited to, poisons, toxins, venoms, harmful chemicals, drugs, heavy metals, other pollutant or any other toxic substances, as are known within the art. The system/method of the present subject matter, utilises the various differences in the refractive index of these toxic substances, in order to differentiate them from the portions of the biological entity, by providing a clear picture regarding a rate-of-spread (between consecutive scans/detections) and the affected-areas by these toxins, within the biological entity without using an intrusive methods, as in the past. Further, the “alien objects” refers to any kind of foreign entities, or, other non-living objects that enter the body of a biological entity, either accidentally or intentionally causing harm to the body or otherwise require medical intervention. These alien objects are those foreign entities, including but not limited to, swallowed objects, inhaled particles, surgical sponges left inside the body, micro-plastics, bullets, shrapnel, glass/metallic/ceramic pieces/shards, or any other non-native material or any other kind of foreign entities as known within the art, that should not be present within the biological entity by default/naturally. Lastly, in one of the embodiments of the present subject matter, the foreign entity is specifically, a port-a-cath, that is detected/identified by the device, allowing the user to insert a syringe-needle into the port-a-cath (that is beneath the skin of a patient/biological entity) in a single-attempt, by assisting them with information regarding the location/position, shape, structure, texture and/or orientation of the port-a-cath, that is disposed within a patient. Enabling the user to successfully puncture the skin and a septum region of the port-a-cath, to ensure painless and comfortable procedure for the patient/biological entity.

Throughout the present subject matter, the term “User” refers to any individual who interacts with and utilizes the see-through device, in order to identify/detect any of the various types of foreign entities that are disposed subcutaneously, within a biological entity and are not visible through the naked eyes. The user hence, is any person, including but is not limited to, doctor, surgeon, medical practitioner, nurse, care-taker, physiotherapist, nutritionist, chiropractor, radiologists, researcher, analyst, coroner, investigator, journalist, food-critique and any other user desiring to see-through (or, rather inside) a biological entity, in order to mainly identify/detect any type of foreign entities and their various specifications/characteristics, such as shape, size, texture, material, orientation and/or most importantly their position/location. Further, in any of the embodiments of the present subject matter, the user is a medical-practitioner/personnel at a hospital, or similar medical facility/institute, where a centralised server network, or the like may exist, allowing for an administrator of the hospital server system, to be able to control several aspects of the system, in order to provide several additional features of the system/method like for example, firstly limiting operational/functional access of the see-through device to only specific personnel/users, and secondly controlling any information communicated to-and-fro between the said device with the external central server/internet, by utilising technologies such as, finger-print sensing, iris-scan, face-recognition, voice recognition, password, pin, ID number, etc., at the device-end, for optimally regulating, securing, logging and maintaining the usage of the various capabilities of the see-through device, by the multitude of users/medical-practitioners at the hospital, while each of them utilises the device for visualizing subcutaneous/visceral portion of a different patient.

Throughout the present subject matter, the term “transmitter-receiver unit” refers to an integrated sub-unit that combines both the transmitting and receiving functions, of the device. Hence, the transmitter-receiver unit is solely responsible for transmitting/emitting signals, waves, photons, beams, etc., towards a biological entity, as well as, more specifically the foreign entities disposed within this biological entity. Then these signals make contact with the various components/parts of the biological entity, as well as, the various foreign entities disposed within, and these get reflected in various directions, at varying angles/frequencies/wavelengths compared to when they were first emitted. Many of these signals get reflected in a plethora of directions, however a fraction of these reflected signals/waves/photons/beams, return towards the original source i.e. the transmitter-receiver unit. This fraction of reflected signals, are then received/absorbed by the transmitter-receiver unit. This process continues for a few seconds to minutes, until eventually after a while, the device begins to paint a picture (in laymen terms, in actuality the device plots/records detection as points in space, until eventually a 3D model is formed), regarding the subcutaneous/visceral portions of the biological entity including the various foreign entities in that region of the body of the biological entity, by calculating the difference in frequencies between the transmitted signals and the received signals, in order to determine several aspects, such as distance, shape, size, texture, material, orientation, and/or most importantly their position/location individually, for each entity and their various parts/portions/components. Further, in one of the embodiments, the transmitter-receiver unit is embedded onto the see-through smart device, in which the transmitter portion of the transmitter-receiver unit, will transmit a multitude of signals, which will then travel through air followed by a biological entity, eventually reaching a foreign entity, as well as, the various subcutaneous/visceral/entrail regions of the biological entity, then some of these signals will get reflected from all these various entities and will traceback their path in order to travel back to the receiver portion of the transmitter-receiver unit, that will then be absorbed/received to be then, filtered, amplified, converted, detected/identified and processed to generate an image/structure of the biological entity and foreign entities that are disposed within the biological entity. This is then presented to the user by the augmented-reality interface, by overlaying this processed information on the display of the see-through smart device, importantly providing a clear-differentiation between both the biological entity and the various identified/detected foreign entities. Furthermore, in some of the embodiments of the present subject matter, the transmitter-receiver unit is versatile in its application and is integrated into one or more separate companion device/s, that are physically separate from the see-through device, that may be considered as portable detection ensembles in such cases, as per some of the embodiments of this method/system of the present subject matter. These companion devices with these embedded transmitter-receiver unit/s, provide an alternate detection device for portable use, mainly for flexibility, as well as, for detecting/identifying entities that are very deep inside the biological entities, by placing these companion device close-enough to the body of the biological entity, in order to avoid/minimize/reduce any kind of noise/distortions/loss that is caused due to an air-medium, that ordinarily exists in-between these two items/objects/entities (the companion device and the biological entity). Since, each of this companion device is a physically separate device in such cases, each of them exclusively communicates over a short-range with the see-through smart device, or in some scenarios with each other, in order to relay the obtained raw-data for processing and consequently presenting this processed information to the user. This flexibility allows medical professionals to choose the most convenient and effective method for foreign entity detection, based on the specific medical scenario. Moreover, in one of the embodiments, the transmitter-receiver unit is capable of being calibrated/adjusted/fine-tuned to detect/identify specific foreign entities that have various distances, sizes, shapes, materials, orientations and textures, as per the specific requirements of the user, as desired. This calibration involves configuring the unit so that, it accurately identifies and responds to specific characteristics of the foreign entities, such as their distance, dimensions, textures, shapes, sizes and/or the materials that they are composed of. This process ensures that the transmitter-receiver unit precisely and reliably detects foreign entities, regardless of their varying forms. For example, foreign entities that vary significantly in size significantly are successfully detected/identified utilising this method/system, from very tiny specific microbial cells, all the way to larger medical implants. Further, these foreign entities may be made from a wide range of materials, including metals, glass, plastics, ceramics, composites and other materials that may be detected this way, as are known within the field, by primarily calibrating the transmitter-receiver unit, in order to tailor the unit for the specific purposes as desired, during their manufacturing/production process. The system in some of the embodiments, is even set up to recognize, and quickly determine these diverse types of foreign entities effectively, maintaining high accuracy and effectiveness as time passes, due to its features of historical-record keeping. Additionally, in yet another embodiment, the transmitter-receiver unit operates in multiple modes, such as continuous scan mode and pulse mode, in order to optimize power consumption and detection performance. Specifically, within continuous scan mode, the transmitter-receiver unit constantly emits/sends signals and listens/receives for responses without any interruption, leading to higher performance, which is ideal for critical applications, such as, for instance during surgical procedures, or, in high-security environments. Similarly, within pulse mode the unit emits/sends signals though only in short/intermittent bursts and then, listens/receives for responses at certain pre-defined intervals, between these bursts/pulses. This flexibility to choose the most appropriate mode, allows users to optimize the energy efficiently, with respect to the particular detection/identification requirements, enhancing the overall functionality and efficacy of the system/method of the present subject matter. Specifically, in one more embodiment of the present subject matter, the transmitter-receiver unit includes an auto-calibration feature that maintains optimal performance, without the need for any kind of a manual intervention. This feature ensures that the system remains accurate and reliable over time, reducing the need for frequent manual adjustments. Lastly, in one of the embodiments of the present subject matter, the transmitter-receiver unit is designed to minimize electromagnetic interference, with respect to other medical equipment. This design/architectural consideration ensures that the system operates effectively without disrupting, or, being disrupted, by other medical devices, maintaining a safe and functional clinical environment, in the vicinity of the transmitter-receiver unit. Ultimately, in any of the embodiments of the present subject matter, the transmitter-receiver unit operational frequency, type, range, etc, includes but is not limited to, Infrared, Near-Infrared, X-rays/CT (computed tomography), MRI (magnetic resonance imaging), MWT (microwave tomography), Ultrasound/Ultrasonic, fluoroscopy, PET (positron emission tomography), endoscopy, arthroscopy, angiography, lymphangiography, mammography, venography, duplex ultrasound, elastography, OCT (optical coherence tomography), EIT (electric impedance tomography), photoacoustic imaging, sonography/radiology, ESI (electrical source imaging), spectroscopy techniques (such as but is not limited to, infrared Spectroscopy, Raman Spectroscopy, Fluorescence Spectroscopy, and most importantly Surface-enhanced Raman Spectroscopy techniques), ultraviolet radiation/frequencies, and several other frequencies (that are time-varying, modulated, polarized, etc.). This wide range of operational frequencies ensures that the system is capable of effectively detecting various types of foreign entities under different conditions, providing robust and versatile detection capabilities. For instance, many of these frequencies are particularly useful, for deep tissue penetration, by providing detailed images of various foreign entities that are situated deeper within the body of a biological entity. It would be obvious to the person skilled in the art that, many of the scanning techniques described above be incorporated into separate portable detection ensembles (or, in other words companion units), in order to realize the various functionalities of several embodiments of the present subject matter. In yet another embodiment of the present subject matter, the various transmitter-receiver unit/s formed by various components, such as but not limited to, lamps, LEDs, acoustic generators, semiconductors, relays, sensors, sensor-arrays, transmitters, receivers, emitters, detectors, signal processing modules, etc. are designed, configured into the architecture based on the specific needs of the user/administrator, particularly.

Throughout the present subject matter, the term “companion device” refers to an additional/auxiliary electronic device, having at least one transmitter-receiver unit, in order to detect/identify any/all types of unmodified foreign entities disposed within a biological entity and provide this obtained raw-data, to the see-through device, so that a processed information may be presented in real-time, by overlaying the same onto the user's field-of-view. The companion device communicates over a short-range with the see-through device, in order to relay all such obtained raw-data, either wirelessly, or, using a wired connection (USB, Type-C, Power/Data Cables/Cords, etc.), utilising any of the known means of such communication channels. Further, in some embodiments the companion device, is an integral portion of the system and is detachably attached to the see-through device. This enables the companion device to detect/identify and obtain raw-data, while still being attached to the see-through device. While, also having the option in cases, when a high precision detection/identification of certain specific type of foreign entities, requires the transmitter-receiver unit to be very close, for mainly improving the detection/identification resolution quality (as it is well known within the field that, the lesser the distance between a transmitter-receiver unit and a target, the higher are the chances that the transmitter-receiver will detect/identify a reflected signal from that target), then, in such cases the companion device may be detached and placed closer to the biological entity, for enabling such improved visualization of the subcutaneous/visceral regions/parts/portions/components/contents of the body of the biological entity, including specifically the various foreign entities disposed within. Furthermore, in some other embodiments, this companion device that is detachably-attached, or, otherwise is a completely separate companion device (having no attachable-detachable capability, with respect to the see-through smart device), may be designed to have a/an opening/receptacle/clip, that is capable of engulfing, receiving, or, attaches to a hoober needle for instance. For example, in case when the foreign entity is a port-a-cath, this separate companion device attaches to a hoober needle detachably, allowing for the user to maintain a free hand (which may be used to stabilise the patient, instead of holding a companion device), while inserting this hoober needle into the subcutaneous port-a-cath of a patient, as well as, enabling this companion device to get a close-up scan of the area by design. Moreover, in some of the embodiments of the present subject matter, the see-through smart device maintains communication with a plurality of physically-separate companion units (regardless of whether, they are capable of attaching detachably with the see-through smart device, or not), while also having a provision to detachably attach some of these companion devices at different locations on the body of the see-through device, allowing for a higher perspective scan to be captured, as multiple transmitter-receiver units located within these multiple companion devices, obtain a far more optimized perspective raw-data, that in turn significantly assists in generating and presenting, a higher order of 3-dimensional processed information resolution, to the user. This multiple companion device architecture, serves other important functions, as in some cases these companion devices are placed on either side of a biological entity portion, then each of these physically-separate companion units may capture/obtain signals transmitted by the other companion unit, obtained through the body of the biological entity after traversing the entire length of the biological entity and everything disposed along the path, as well as, the reflected raw-data as previously disclosed, enabling the see-through device to get even more significantly higher resolution of raw-data from the various transmitter-receiver units of these companion devices, for generating the most qualitative processed information, to provide the absolute best results. This multiple companion device architecture is scaled-up for situations that demand very degrees of precision, by utilising more than two, or, sometimes even three companion devices simultaneously, in order to receive, each other's transmitted signals from various different locations/positions (depending on the total number of companion devices involved in the scenario), in order to generate multiplexed, convoluted raw-data of all the identified/detected entities. Such companion devices, in some scenarios be scanning using the same techniques, or, in other instances utilise completely different scanning frequencies/techniques, in order to obtain/acquire all the pertinent/necessary raw-data, to then generate the most optimal/precise processed information to the user. It is also important to note that, each of the companion devices, firstly contain their own set of internal electric/electronic components in order for them to be able to perform all the above disclosed processes, as well as, each of these companion devices is made into various shapes, sizes, articles, including but not limited to, horseshoe, square, rectangle, oval, elliptical, cardioid, or any other shape or size as known within the art, as per the user preference, or, alternately depending on which shape/size enhances the detection/identification efficiency, or, the overall operability of the system/method of the present subject matter. It is also understood by a person skilled in the art that, in some embodiments where a companion device does not detachable attach to a see-through smart device, a wired companion device is connected with via either, a cable/cord with the see-through smart device itself, or, this companion device sports a plug-in power-plug that can siphon power from a nearby electric/wall socket while communicating wirelessly with the see-through smart device, hence eliminating the need for the companion device to have a power storage unit of its own, in some such embodiments of the present subject matter, to further reduce the cost of the overall system. Lastly, in some embodiments of the present subject matter, the see-through smart device and the plurality of companion devices, have a combined physical shape, that is akin to the face of an animal, bird, fish, fictional-characters, celebrities, cars, dolls, toys, etc. This feature is primarily incorporated into the design of the system/method, in order to assist in calming and engaging paediatric patients especially, as for example, in these embodiments the design of these devices is structured such that, the see-through smart device forms the main portion of any of these faces as described above, while the various companion devices form parts of the face, such as but is not limited to, ears, eyebrows, hands/palms, or even a nose, of this face shaped see-through device. In such cases, the outer shell of these devices may sport various organic/non-organic materials, to imitate organic features, such as but is not limited to, skin, hair, fur, leather, suede, etc. for haptic comfort. This kind of a design of the system allows the biological entity (especially child patients, or, timid entities/animals) to not be fearful of the see-through smart device worn by the user, or, the companion device (when it is detached and brought closer to the patient), and in some cases may even encourage these biological entities to actively/willingly assist the user/medical-practitioner with a procedure/detection, by holding this attractively shaped/textured companion device close to their body, while also catching the attention of these patients/biological entities, to keep them engaged for the duration of the procedure. Finally, the companion devices, each employs the same/different type of transmitter-receiver unit, compared to the other companion device, when multiple companion devices are included by the system/method, in an embodiment of the present subject matter. Ultimately, within any of the embodiments of the present subject matter, the companion device, has a plurality of buttons/haptic-sensors (to turn it on/off, changing scan-mode, checking power left in the device), as well as, visual indicators (for e.g. LED lamps, audio-ping, etc.) for indicating the current battery-level, alignment with the biological entity being detected/identified, etc. as may be required/desired by the user. It is also essential to note that, each of the companion devices get electrically charged, while they are attached to the see-through smart device, in any of the embodiments of the present subject matter. It should be noted that, in certain embodiments of the present subject matter, the see-through smart device is capable of detachably attaching with several companion devices, wherein these companion devise each may operate using different type of scanning techniques, this allows the user to take advantage of the system in a very interesting way, in that he/she may first obtain a processed information by utilising one type of companion device (for e.g. ultrasound scanning), then he/she may proceed to detach this companion device and instead attach another type of companion device (identical in shape/size to the previous companion device) that utilises Near-Infrared frequency for scanning, to then obtain this new form of raw-data, to then proceed to collate the previously acquired ultrasound raw-data and this newly obtained live NIR raw-data, to form a highly precise processed information, for the user. Hence, allowing the user in one instance to practically be able to swap companion devices, as easily as, changing a cassette in a cassette player, or akin to, replacing game cartridges in a game boy, for example. Retrospectively, in some embodiments of the present subject matter, the companion device, is detached from the see-through device during detection/investigation process, to then be temporarily coupled to a magnification/enhancement equipment, which increases the strength of the companion device, in terms of visualization capabilities. This magnification/enhancement equipment, is digitally communicative with the companion device/see-through smart device, and primarily provides the companion device with extra power, additional transmitter-receiver unit data from the magnification/enhancement equipment, magnify/amplify the signal strength of this companion device, additional transmitting/receiving surfaces in order to capture more amount of reflected signals from the biological entity, (including the foreign entities that are disposed within the biological entity), providing expandable reflective/interactive surfaces/elements (such as, but is not limited to, lenses, mirrors, reflective elements, and other elements that utilise microscopic/telescopic techniques of EM wave enhancement/magnification) that are temporarily placed around the specific region of the biological entity (enhancing the capability of this companion device by allowing the receiver portion of the transmitter-receiver unit of the companion device to accumulate more number of reflected signals/frequencies from the biological entity and the foreign entity disposed within it, that are otherwise lost in other embodiments of this present subject matter, thus providing the most efficient method of implementing this present subject matter), and/or other known enhancement/magnification architectures, as known within the art.

Throughout the present subject matter, the term “internal electronic components” refers to the various core components, that enable the system and method as described within the present subject matter, to perform all the described functions/processes. These core components include components such as, but not limited to, memory units, central processing units (CPU), graphics processing units (GPU), power storage units, amplification units, communication units, secondary electronic components, transmitter-receiver units, electromechanical components, cameras, or, any other components as required by the see-through smart device, or, the various types of companion devices, as is known within the art. These components work together to ensure the device operates efficiently and effectively under the commands received from a software application. It is also understood by a person skilled in the art, that depending on the specific requirements of the user, a plurality of each of these components, is employed by either of the above described devices, in order to accomplish the various feats and applications of the system/method, that are described within the present subject matter.

Throughout the present subject matter, the term “memory unit” refers to a critical component of the smart/companion device responsible for storing data, programme, software, firmware, interface, protocols and other instructions necessary for the device's operation. Further, the “memory unit” consists of both volatile and non-volatile memory. The volatile Memory (RAM) is mostly utilised for temporarily storing data/information, or to cache parts of data, that is/are actively being used or processed by a CPU. The non-volatile Memory (e.g., Flash Storage, HDD, SATA) retains data even when the device is powered off, hence it is used for long-term storage of the operating system, applications, historical-data, local profiles/EHR and other user-data, or, even entity-detection related data/information for future reference and analysis.

Throughout the present subject matter, the term “Central Processing unit (CPU)” refers to the primary component that performs most of the processing, within the see-through smart device, or, any one of its counter-part companion devices. The CPU executes instructions received from the software application and manages all the activities of all other hardware components of the device, optimally and efficiently. Further, the CPU performs arithmetic, logical, control, and input/output (I/O) operations specified by the instructions provided to it, from time to time. In some instances, the CPU, includes several cores, each capable of executing instructions independently, enhancing the device's multitasking capabilities.

Throughout the present subject matter, the term “Graphics Processing unit (GPU)” refers to a specialized processor, designed to accelerate the creation and rendering of images, videos, and animations. Further in some instances, the GPU contains a large number of smaller cores optimized for parallel processing, which allows it to handle multiple tasks simultaneously, as well as, enabling it to provide a higher-resolution of processed information, in real-time. Furthermore, the GPU is an essential component these days, for any kind of applications requiring high graphical performance, such as but not limited to, virtual/augmented reality display, graphical user interfaces, visual/graphical data processing, or, any other data-heavy visual applications/modules, as known within the art.

Throughout the present subject matter, the term “Power Storage unit” typically refers to a battery that stores electrical energy and supplies this power to the device (see-through smart device, or, companion device) and its various internal components. Further, the power storage unit may also include circuitry that enable, power management of the device, viewing of battery usage, controlling the charge/discharge cycles, battery health monitoring, etc., in order to regulate the distribution of power securely and ensure efficient energy usage, by the various components of the system/method.

Throughout the present subject matter, the term “Amplification unit” refers to a unit, that primarily increases the power of a signal, such as an audio or RF signal, to ensure the enhancement of useful portion of the raw-data from the obtained signals, for effective processing of the same and increasing the accuracy of the overall system/method. The components of the amplification unit, including but not limited to, amplifiers and/or transistors that boost the signal strength. This unit is crucial for maintaining the quality and clarity of obtained signals/raw-data, and for ensuring effective interpretation of the various raw-data obtained from the various types of entities, such as the biological entity, or, the foreign entities, as described herein.

Throughout the present subject matter, the term “Communication unit” is a unit that enables the device to send and receive various types of data/information through various means of communication. The communication units consist of either wired/wireless communication units. Further, the wired communication provides high-speed and reliable data/information transfer through physical connections, that can operate on various interfaces, including but not limited to, USB, Type-C, Ethernet, HDMI, LAN, WAN, Bus-Cables, power/data cable/cord, or any other similar physical interface, as known within the field. Furthermore, the wireless communication allows the device, to connect to other devices and networks without the use of any physical cables and utilises various technologies, including but not limited to, Wi-Fi, Bluetooth, Radio-frequency, NFC, Li-Fi, Lo-Ra, Infrared, IOT, cellular networks, or, any other similar non-physical technology, as known within the field. In some embodiments, it is important to note that, the transmitter-receiver units of multiple companion devices, is/are designed to communicate wirelessly with the see-through smart device. This preferable wireless capability enhances the system's ease of use and flexibility, allowing for seamless data transmission and reducing the need for cumbersome cables.

Throughout the present subject matter, the term “secondary electronic components” is the additional components that support the primary functions of the device, enhance the device's functionality and user experience, these include components, such as but not limited to, sensors (e.g., accelerometers, gyroscopes), connectors (e.g., USB ports, headphone jacks), relay, motherboards, capacitors, resistors, safety circuits, power-break circuits, over-ride circuits, inductors, wiring, switches, IC, modules, or other any other component as required by the see-through smart device, or the companion device in order to accomplish their respective functions/processes as described herein, as are known within the art.

Throughout the present subject matter, the term “electromechanical components” are the components that combine the electrical and mechanical processes to perform mechanical actions in response to electrical signals, or vice-versa, enabling physical interaction and feedback with/to the see-through smart device. These electromechanical components include components, such as but not limited to, motors, switches and actuators, or the like.

Throughout the present subject matter, the term “camera” refers to an optical device, that captures and records real-time images/video, of the biological and foreign entities which are stored, displayed, and/or processed. This is definitely an optional integration, that essentially, enhances the system's ability to document procedures, assist in further analysis, and provide a comprehensive visual record of biological entity/foreign entity. As well as, the camera also assists in orienting the overlaid processed information, to be precisely laid over the body of the biological entity that is in front of the user's field-of-view, while the camera itself may have a low resolution, as it has very little effect on its abilities, to effectively assist the system/method with these above described aspects. Further, in some embodiments of the present invention, a separate video camera that is in communication with at least the see-through smart device, is integrated into the system, as an additional companion device.

Throughout the present subject matter, the term “software application” refers to a comprehensive set of programs designed to operate within the see-through smart device/companion device, that employs various algorithms/programmes/modules/models, such as, but not limited to, manual, Artificial Intelligence (AI), generative AI, Machine Learning (ML), Large Language Models (LLM), Reinforcement Machine Learning (RML), Neural networks, Convolutional Neural Networks (CNN), and other known complex-algorithms within the art, that are utilised for generating the processed information. The software application processes the obtained raw-data received from the transmitter-receiver unit, and integrates it onto the user's field-of-view, enabling real-time, adaptive visualization of the foreign entities disposed within the body of the biological entity. In yet another embodiment, the software application includes several application modules, such as, but not limited to, battery management, gaze tracking, customizable user interface (a user interface that allows customization of visualization settings, based on user preferences and specific medical procedures), updatable firmware, and other known application modules, as are known within the art.

Below provided are the major embodiments of the present subject matter, in order to provide context to the drawings, as well as the claims, provided within this patent application.

### Exemplary Embodiments of the System and Method for Analysing Rating-Based Impact, Are Defined Below

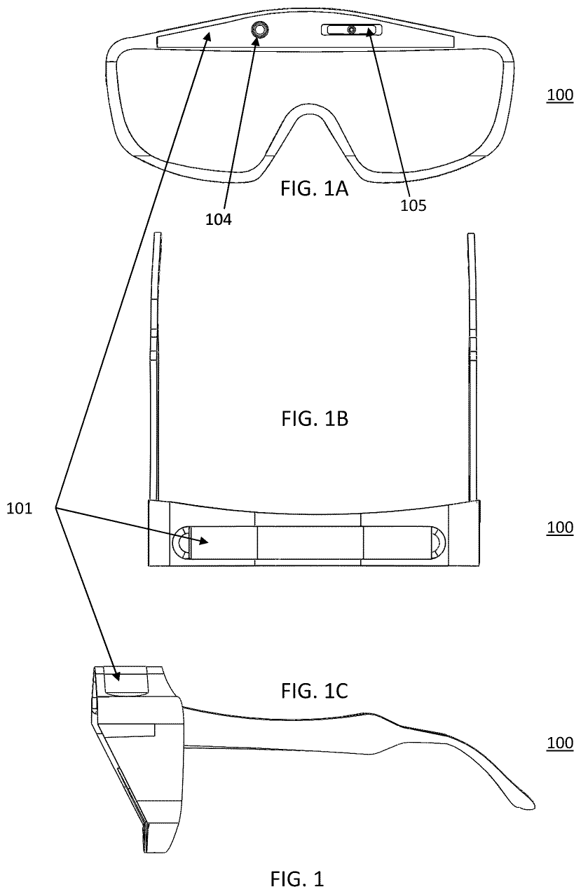

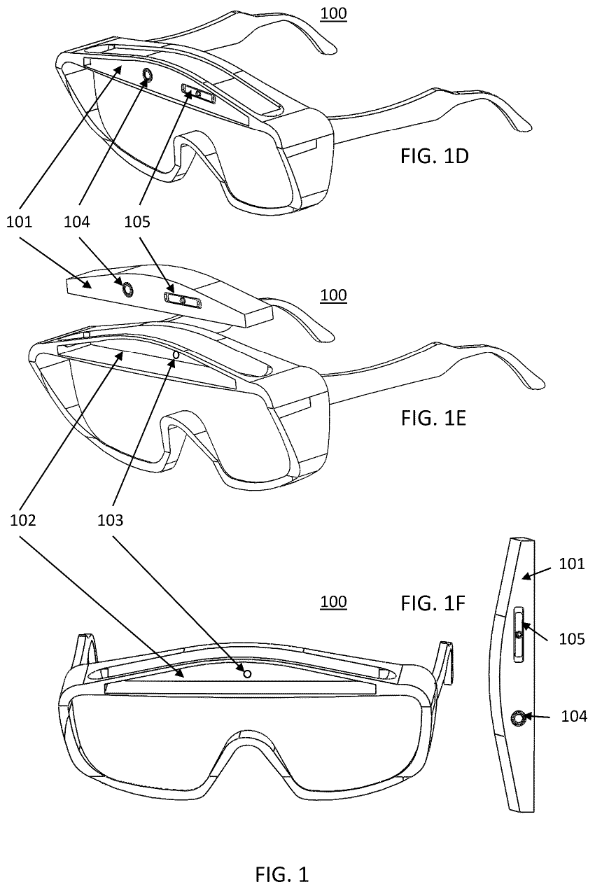

In a first exemplary embodiment of the present subject matter, as disclosed within FIG. 1. It illustrates a primary device embodiment of the system and method for subcutaneous visualization, comprising a see-through smart device having a detachable companion device, that has been depicted in various views/orientations, herein.

Specifically, as disclosed within FIG. 1 of the present subject matter, is a see-through smart device 100, that provides the processed information to a user. This see-through smart device 100 also has a slot/receptacle 102 along the top-portion in this embodiment, for detachably attaching a companion device 101. Further, the see-through device has a dedicated camera 103, located on the anterior-wall of the slot/receptacle 102, that provides visual co-relation with respect to the companion device, whenever the companion device is detached for a close-up detection/inspection of a biological entity of interest.

The FIGS. 1A, 1B and 1C, depict a front-view, a top-view, and a side-view of this see-through smart device 100, respectively, wherein the detachable companion device 101 is still attached to the see-through smart device 100, being disposed within the slot/receptacle 102, as shown, while also covering the camera 103, as there is no requirement to utilise this camera, while the companion device is still attached to the see-through smart device, as the companion device also has an additional camera 104 in this embodiment, along with a dedicated transmitter-receiver unit 105.

Further, the FIGS. 1D and 1E depict, a perspective view of the see-through smart device 100. Here, the FIG. 1D depicts that the companion device 101 is still attached to the see-through smart device 100, while the FIG. 1E depicts that the companion device 101 is detached/slid-out from/of the slot/receptacle 102 of the see-through smart device 100. This companion device 101 communicates with the see-through smart device 100 wirelessly, when detached. Further, while the companion device 101 is attached to the see-through smart device 100, it facilitates hands-free operation, as well as, switches to a physical communication mode (turning-off wireless communication for the duration of attachment), in order to realise the various functions/processes, as described within this present subject matter of the system/method.

Furthermore, the FIG. 1F depicts, the see-through smart device 100 and the companion device 101, side-by-side in a completely detached orientation.

However, it should be noted that, in some other embodiments (not illustrated) of the present subject matter, the see-through device has the camera 103 at a different location on the see-through smart device, which is not hindered by the attachment/detachment of the companion device, in order to always keep recording/monitoring the procedures performed by the user. Similarly, in other embodiments (not illustrated) of the present subject matter, the companion device has a wired communication architecture with the see-through smart device, instead of a wireless communication architecture as described in FIG. 1. Also, in yet other embodiments (not illustrated) of the present subject matter, the companion device has a more than one transmitter-receiver unit 105, wherein these plural transmitter-receiver units may or may not operate in the same frequency range with respect to each other, contrary to the components as described in FIG. 1.

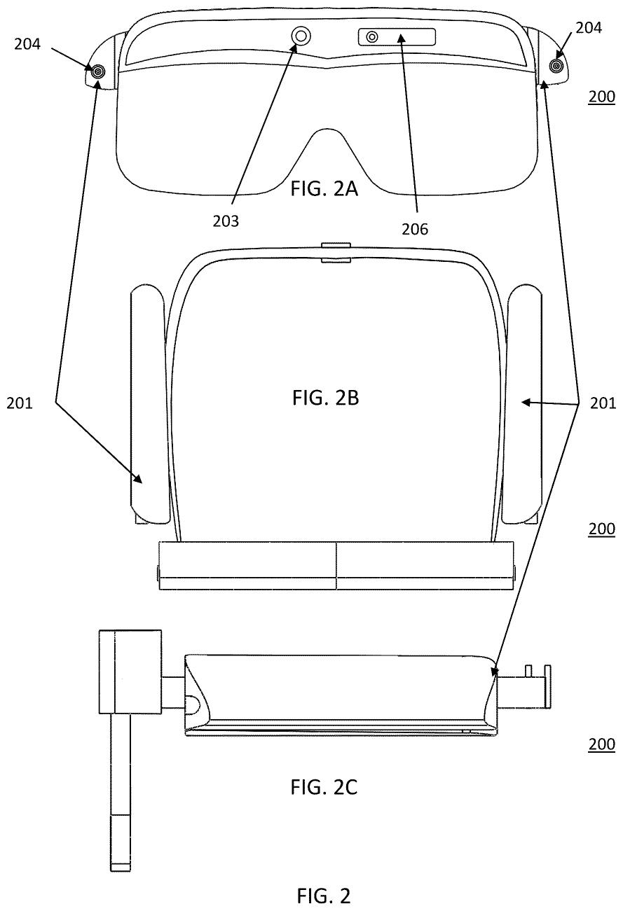

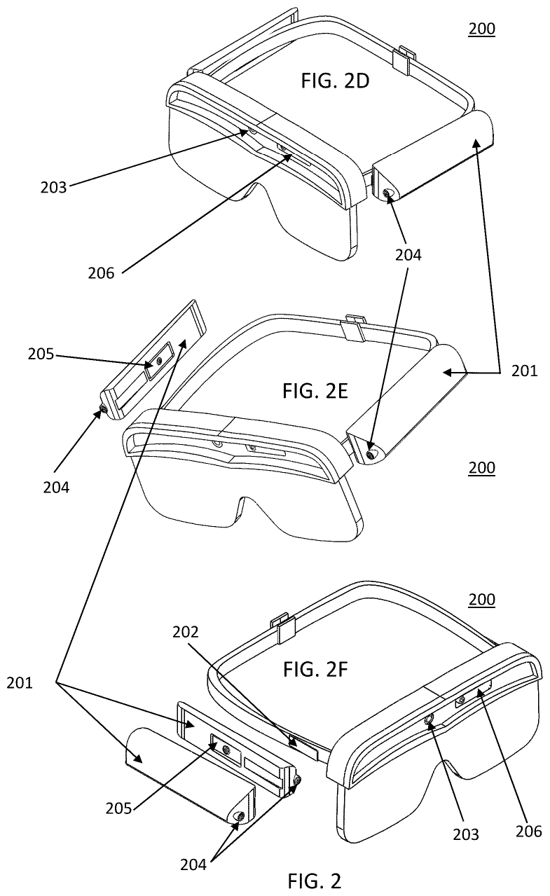

In a second exemplary embodiment of the present subject matter, as disclosed within FIG. 2. It illustrates a primary device embodiment of the system and method for subcutaneous visualization, comprising a see-through smart device having a plurality of detachable companion devices, that have been depicted in various views/orientations, herein.

Specifically, as disclosed within FIG. 2 of the present subject matter, is a see-through smart device 200, that provides the processed information to a user. This see-through smart device 200 has a plurality of slots/receptacles 202 along the belt-portion in this embodiment, for detachably attaching a plurality of companion devices 201. Further, the see-through device has a dedicated camera 203 and a dedicated transmitter-receiver unit 206, both of which are located on the front-central portion of the see-through smart device 200. The dedicated camera 203 provides visual co-relation, with respect to the companion device, whenever the companion device is detached for a close-up detection/inspection of a biological entity of interest, as well as, the dedicated transmitter-receiver unit 206 provides a basic capability of identifying/detecting the various foreign entities, without even having to turn-on the companion devices, in some instances.

The FIGS. 2A, 2B and 2C, depict a front-view, a top-view, and a side-view of this see-through smart device 200, respectively, wherein the detachable companion devices 202 are still attached to the see-through smart device 200, being disposed within the slot/receptacle 201 provided on the belt of the see-through smart device, as shown, while the companion device 201 also has an additional camera 204 in this embodiment, along with another dedicated transmitter-receiver unit 205.

Further, the FIGS. 2D and 2E depict, a perspective view of the see-through smart device 200. Here, the FIG. 2D depicts that the companion devices 201 are still attached to the belt of the see-through smart device 200, while the FIG. 2E depicts that the companion devices 201 are detached/slid-out from/of the slot-receptacle 202 on the belt of the see-through smart device 200. These companion devices 201 communicate with the see-through smart device 200 wirelessly, when detached. Further, while these companion devices 201 are attached to the see-through smart device 200, it facilitates hands-free operation, as well as, switches to a physical communication mode (turning-off wireless communication for the duration of attachment), in order to realise the various functions/processes, as described within this present subject matter of the system/method.

Furthermore, the FIG. 2F depicts, the see-through smart device 200 and the companion devices 201, side-by-side in a completely detached orientation.

However, it should be noted that, in some other embodiments (not illustrated) of the present subject matter, the see-through device has the camera 203 and the transmitter-receiver unit 206, at a different location on the see-through smart device, regardless of being hindered by the attachment/detachment of the companion devices, in order to always keep recording/monitoring the procedures performed by the user. Similarly, in other embodiments (not illustrated) of the present subject matter, the companion devices have a wired communication architecture with the see-through smart device, instead of a wireless communication architecture as described in FIG. 2. Also, in yet other embodiments (not illustrated) of the present subject matter, the companion devices have more than one transmitter-receiver unit 205, wherein these plural transmitter-receiver units may or may not operate in the same frequency range with respect to each other, contrary to the components as described in FIG. 2. Further, the transmitter-receiver units 205 of each of these companion devices, operate within different wavelength/frequency ranges compared to each other, in order to enhance detection/identification capabilities, in some embodiments of the of the system/method as described by the present subject matter. Furthermore, in further other embodiments (not illustrated) of the present subject matter, there are more than two companion devices that are in communication with the see-through smart device, and these companion devise provide various types of obtained raw-data and/or processed information, to the see-through smart device.

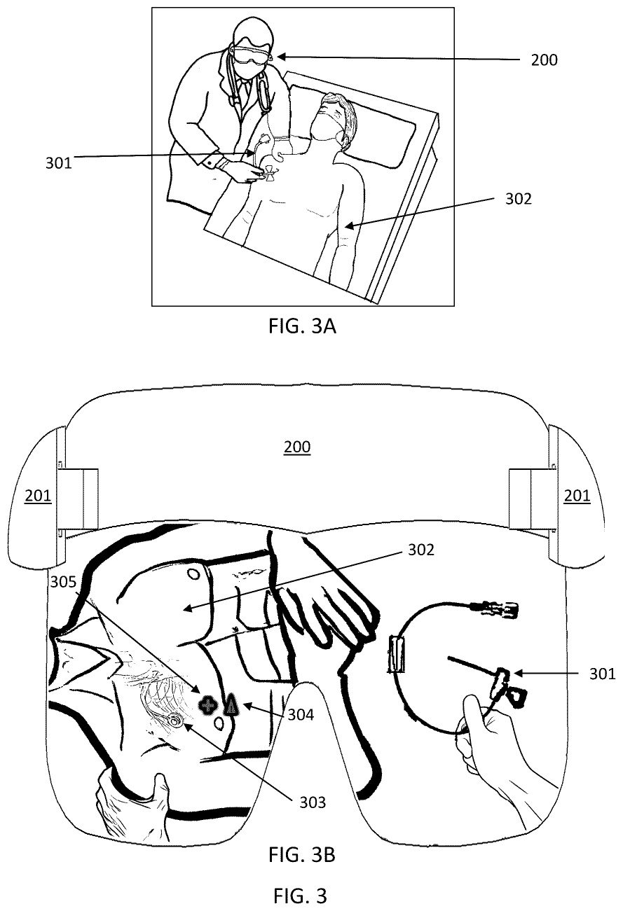

In an exemplary embodiment of utilising the system/method of the present subject matter, as disclosed within FIG. 3. It illustrates, one embodiment of the many medical/research area/domains/fields, where this system and method of subcutaneous visualization, utilises a see-through smart device.

Particularly FIG. 3A, of the FIG. 3 illustrates, a user/medical-practitioner utilising a see-through smart device 200 (from, FIG. 2), for the purpose of visualizing subcutaneous/visceral portions of the patient/biological entity, in real-time. While the prime objective of the user here, is to identify/detect the presence of a foreign entity (or, more particularly, a medical implant, in this case) with the help of the see-through smart device 200, as clearly this medical implant (e.g., port-a-cath) is disposed inside the patient's dermal layers, and hence is not visible to the user when viewed with naked-eyes.

Further, the FIG. 3A clearly shows a user wearing a see-through smart device 200, while he is holding a needle (here, a Huber-needle) 301 and gazing at the chest-portion of a patient 302. As seen here the user, cannot see where the medical implant, namely the port-a-cath is located within the patient 302, with his naked eyes. Further, as known within the field, a port-a-cath has a septum portion, which forms the portion of any port-a-cath, that is penetrable by a Huber-needle 301, in order for the user/medical-practitioner to access and administer drugs, chemicals, other intravenous injections, or, to draw blood-samples, etc, as known within the field. Here, the see-through smart device 200, provides necessary/important/crucial information, that is particularly the details regarding, where the foreign entity is located/positioned, as well as, the shape, orientation, texture, material, etc. of this foreign entity, by overlaying this information on the user's field-of-view, all the while also providing a clear-differentiation (visually/auditorily) to the user, in-between the detected/identified foreign entity, and the various visceral/subcutaneous contents/components of the biological-entity/patient.

Furthermore, the FIG. 3B depicts a perspective view, as seen by the user/medical-practitioner, with respect to the scenario, as set-forth by the FIG. 3A. This FIG. 3B illustrates a point-of-view screenshot of the see-through smart device 200 that is worn by the user, before the user has initiated the medical procedure. It is clearly seen within this FIG. 3B that the user is seeing the processed information on the display of the see-through smart device 200, while he is holding a needle (here, a Huber-needle) 301 in his right hand, and is still gazing at the chest-portion of a patient 302, who has a subcutaneous port-a-cath 303 within him. Here it is seen that, the port-a-cath 303 is visually presented to the user with the clear-differentiation, as well as, the device also presents visually to the user, a visual representation of all the nearby critical portions of the patient 302. Moreover, in this embodiment a clutter-free background-contrasted visual item is being presented to the user, more particularly a “caution” sign/symbol 304, to alert that the medical implant has been identified and that caution must be taken, near that region/area, during the impending medical procedure, that involves piercing the port-a-cath 303 using the Huber-needle 301. It is also, important to note that, the system/method provided by the present subject matter, allows this user to use their other hand (left-hand, here), in order to stabilise the patient during this procedure, which makes the entire procedure easy, comfortable and quick for the patient 302, while only enduring the absolute least amount of pain caused due to the needle-piercing action (as, this is now easily accomplished in a single-try).

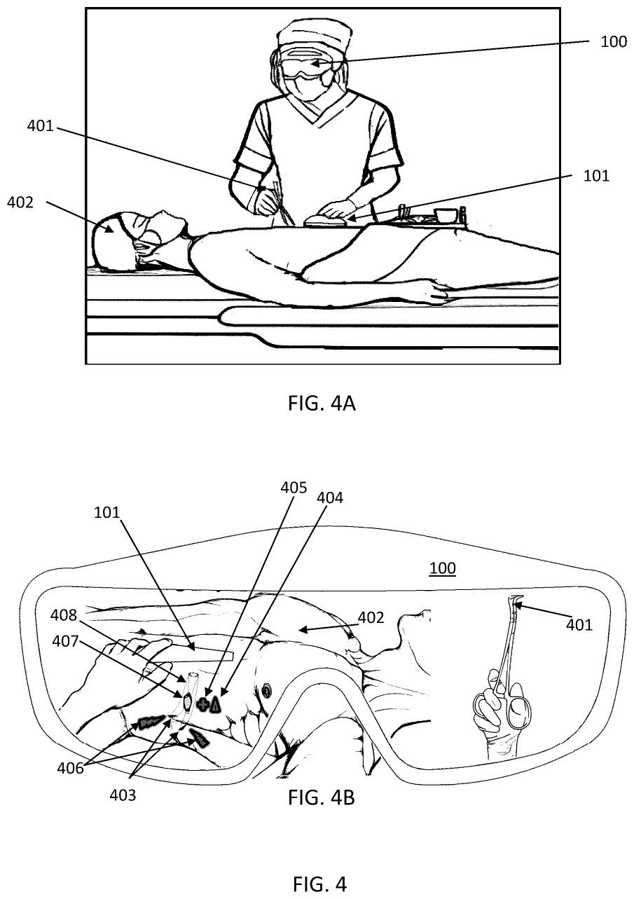

In an exemplary embodiment of utilising the system/method of the present subject matter, as disclosed within FIG. 4. It illustrates, one embodiment of the many medical/research area/domains/fields, where this system and method of subcutaneous visualization, utilises a see-through smart device.

Particularly FIG. 4A, of the FIG. 4 illustrates, a user/medical-practitioner utilising a see-through smart device 100 (from, FIG. 1), for the purpose of visualizing subcutaneous/visceral portions of the patient/biological entity, in real-time. While the prime objective of the user here, is to identify/detect the presence of a foreign entity (or, more particularly, an alien object, in this case) with the help of the see-through smart device 100, as clearly this alien-object (e.g., bullet fragments) is/are disposed inside the patient's dermal layers, and hence is not visible to the user when viewed with naked-eyes.

Further, the FIG. 4A clearly shows a user wearing a see-through smart device 100, while she is holding a companion device 101 and a surgical-forceps 401, while gazing at the bullet-wound portion of a patient 402. As seen here the user, cannot see where the alien-objects, namely the bullet-fragments are located within the patient 402, with her naked eyes. Further, as known within the field, a bullet after penetrating the skin-tissues of a body, fragment into several shards sometimes. The user must identify all these bullet-fragmentations/shards and remove them from the body of the biological entity/patient, as soon as possible, for the safety of the patient's life, all the while making sure that she does not accidentally cause any further haemorrhaging, or any other kind of harm to, any of the critical portions of the patient 402, during this procedure. Here, the see-through smart device 100, provides necessary/important/crucial information, that is particularly the details regarding, where the foreign entity is located/positioned, as well as, the shape, size, orientation, trajectory, etc. of this foreign entity, by overlaying this information on the user's field-of-view, all the while also providing a clear-differentiation (visually/auditorily) to the user, in-between the detected/identified foreign entity, and the various visceral/subcutaneous contents/components of the biological-entity/patient.

Furthermore, the FIG. 4B depicts a perspective view, as seen by the user/medical-practitioner, with respect to the scenario, as set-forth by the FIG. 4A. This FIG. 4B illustrates a point-of-view screenshot of the see-through smart device 100 that is worn by the user, before the user has initiated the medical procedure. It is clearly seen within this FIG. 4B that the user is seeing the processed information on the display of the see-through smart device 100, while she is holding a companion device 101 in her left hand that is placed on the body of the patient near the bullet-wound, and is also holding a surgical-forceps (specifically, an appropriate tenaculum) 401 in her right hand, while she continues to gaze at the chest-portion of a patient 402, who has a plurality of subcutaneous bullet-fragmentations 403 within him. Here it is seen that, the bullet-fragments 403 are visually presented to the user with the clear-differentiation, as well as, the device also presents visually to the user, a visual representation of all the nearby critical portions of the patient 402. Particularly, the device highlights a rib-cage bone portion 407 near the identified bullet-fragments 403 that has been damaged, due to the damage inflicted by this bullet injury. Moreover, in this embodiment a plurality of clutter-free background-contrasted visual items are being presented to the user, more particularly a “caution” sign/symbol 404, an “Immediate Medical Attention” sign/symbol 405, and a plurality of “pointer” signs/symbols 406, in order to alert that the bullet-fragments have been identified, caution must be taken near some regions/areas, immediate medical attention is required at certain locations, possible trajectory paths 408, to access the bullet-fragments 403, during the impending medical procedure, that involves removal of the bullet-fragments 403 and conducting surgery near the rib-cage bone portion 407. It is also, important to note that, the system/method provided by the present subject matter, allows this user to visualize a possible safe trajectory 408, in order to safely/securely/quickly remove the bullet fragments 403 from the body of the patient during this procedure. This makes the entire procedure easy, comfortable and quick for the patient 402 while minimizing any kind of, further risk of contamination, unnecessary delays, accidental internal haemorrhaging, medical incisions, etc., as understood within the art. Here, specifically the companion device plays a major role, as it provides a much higher resolution of raw-data, in-turn improving the quality of the final processed information, that is provided to the user. Particularly, the companion-device 101 avoids the air-gap between the biological entity and the transmitter-receiver unit that it employs, in order to obtain such higher degree of detailed raw-data, comparatively with respect to when the companion device was, if for example still attached to the see-through smart device 100, as shown in FIG. 1D, above.

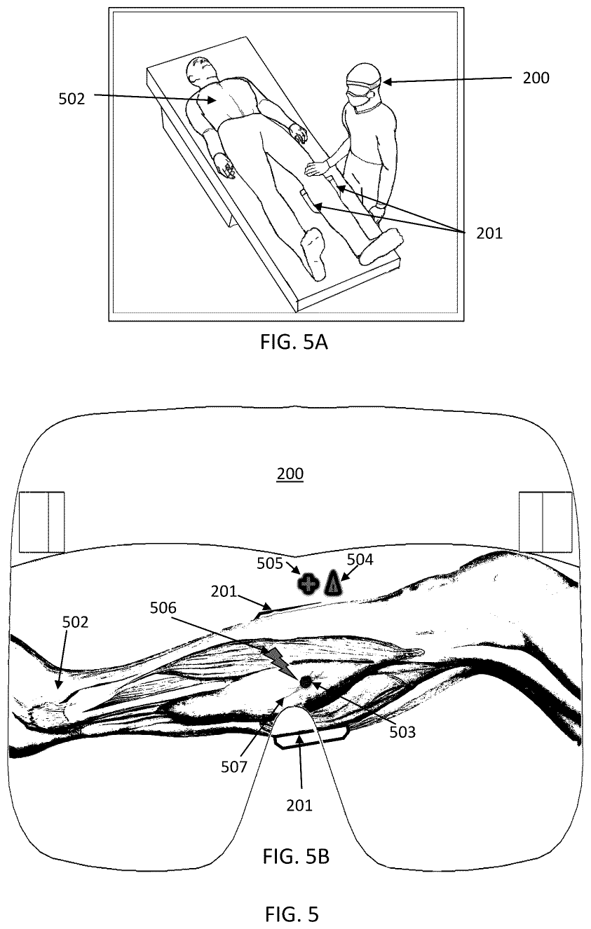

In an exemplary embodiment of utilising the system/method of the present subject matter, as disclosed within FIG. 5. It illustrates, one embodiment of the many medical/research area/domains/fields, where this system and method of subcutaneous visualization, utilises a see-through smart device.

Particularly FIG. 5A, of the FIG. 5 illustrates, a user/medical-practitioner utilising a see-through smart device 200 (from, FIG. 2), for the purpose of visualizing subcutaneous/visceral portions of the patient/biological entity, in real-time. While the prime objective of the user here, is to identify/detect the presence of a foreign entity (or, more particularly, a type of blood-clot, in this case) with the help of the see-through smart device 200, as clearly this blood-clot (e.g., Thrombophlebitis) is disposed inside the patient's dermal layers, and hence is not visible to the user when viewed with naked-eyes.

Further, the FIG. 5A clearly shows a user wearing a see-through smart device 200, while he has placed a plurality of companion devices 201, near a suspected portion of the patient 502, while gazing at the suspected portion of a patient 502, specifically at a location where the patient 502, feels the maximum discomfort due to this blood-clot. As seen here the user, cannot see where the blood-clot, namely the Thrombophlebitis is exactly located within the patient 502, with his naked eyes. Further, as known within the field, a blood-clot such as this, may break-off into several pieces/components and spread through the body, once separated such pieces/components move very quickly through the veins and the rest of the blood-circulation system into the entire body, while endangering several key life-sustaining functions/systems that it encounters, such as but is not limited to, nervous system, circulatory system, respiratory system, cardiovascular system, etc. Hence, the user must identify the blood-clot's location quickly and immediately move to neutralise this Thrombophlebitis, within the biological entity/patient 502, as soon as possible, for the safety of the patient's life, all the while making sure that he does not accidentally cause any further haemorrhaging, or any other kind of harm to, any of the critical portions of the patient 502, during this procedure. Here, the see-through smart device 200, provides necessary/important/crucial information, that is particularly the details regarding, where the foreign entity is located/positioned, as well as, the shape, size, orientation, texture, neighbouring regions, etc. of this foreign entity, by overlaying this information on the user's field-of-view, all the while also providing a clear-differentiation (visually/auditorily) to the user, in-between the detected/identified foreign entity, and the various visceral/subcutaneous contents/components of the biological-entity/patient.

Furthermore, the FIG. 5B depicts a perspective view, as seen by the user/medical-practitioner, with respect to the scenario, as set-forth by the FIG. 5A. This FIG. 5B illustrates a point-of-view screenshot of the see-through smart device 200 that is worn by the user, before the user has initiated the medical procedure. It is clearly seen within this FIG. 5B that the user is seeing the processed information on the display of the see-through smart device 200, while he has placed a plurality of companion devices 201 near a suspected portion of the patient 502, while he continues to gaze at the suspected leg/calf portion of a patient 502, who is suffering the symptoms of Thrombophlebitis, hence the patient 502 requires a procedure of Sclerotherapy in order to overcome this particular ailment. Here it is seen that, the blood-clot (Thrombophlebitis) 503 is visually presented to the user with the clear-differentiation, as well as, the device also presents visually to the user, a visual representation of all the nearby critical portions of the patient 502. Particularly, the device highlights portions of a nearby venous circulatory system 507, that is near the identified blood-clot 503 that has been slowly accumulating in size and mass of the circulatory system within the body of the patient 502. Moreover, in this embodiment a plurality of clutter-free background-contrasted visual items are being presented to the user, more particularly a “caution” sign/symbol 504, an “Immediate Medical Attention” sign/symbol 505, and a “pointer” sign/symbol 506, in order to alert that the blood-clot 503 has been identified, caution must be taken near some regions/areas, immediate medical attention is required to mitigate the situation, nearby trajectory paths that blood-clot 507 may travel through, during the impending medical procedure, that involves neutralization of as much of the blood-clot 503, as is possible, by utilising the sclerotherapy procedure. It is also, important to note that, the system/method provided by the present subject matter, allows this user to visualize a possible blood-clot spreading trajectory 507, in order to enable the user to predict/hinder/alter/disrupt this blood-clot trajectory 507, within the body of the patient during this procedure. This makes the entire procedure easy, comfortable and quick for the patient 502 while minimizing any kind of, further risk of contamination, unnecessary delays, accidental internal haemorrhaging, medical incisions, etc., as understood within the art. Here, specifically the companion devices play a major role, as they provide a much higher resolution of raw-data, in-turn improving the quality of the final processed information, that is provided to the user. Particularly, the companion-devices 201 avoid the air-gap between the biological entity and the transmitter-receiver unit that they each employ, in order to obtain such higher degree of detailed raw-data, comparatively with respect to when the companion devices were, if for example still attached to the see-through smart device 200, as shown in FIG. 2D, above.

While this specification contains many specific implementation details, these should not be construed as limitations on the scope of any implementations or of what may be claimed, but rather as descriptions of features specific to particular embodiments of particular implementations. Certain features that are described in this specification in the context of separate embodiments may also be implemented in combination in a single embodiment. Conversely, various features that are described in the context of a single embodiment may also be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub combination or variation of a sub combination.

Similarly, while methods/operations are depicted in the drawings in a particular order, this should not be understood as requiring that such operations be performed in the particular order shown or in sequential order, or that all illustrated operations be performed, to achieve desirable results. Moreover, only particular embodiments of the subject matter have been described, here. Other embodiments are within the scope of the following claims. In some cases, the actions recited in the claims is performed in a different order and still achieve desirable results. In addition, the processes depicted in the accompanying figures do not necessarily require the particular order shown, or sequential order, to achieve desirable results.

What is claimed is:

1. A subcutaneous visualization system, comprising:
a see-through smart device, for presenting a processed information in real-time to a user;
characterized in that, the processed information provides a clear-differentiation between at least one foreign entity, that is disposed within a biological entity.

2. The system as claimed in claim 1, wherein, the processed information provides precise location, shape, texture, structure and orientation of the foreign entities, with respect to the biological entities.

3. The system as claimed in claim 2, wherein, the biological entity is selected from the group consisting of, mammals, fish, reptiles, amphibians, plants, insects, molluscs and annelids.

4. The system as claimed in claim 2, wherein the foreign entities are selected from the group consisting of silicon, plastic, glass, metals, composites, combinations of these and other known foreign entities.

5. The system as claimed in claim 2, wherein, the foreign entities are selected from the group consisting of, medical implants, pathological entities, traumatological entities, toxicological entities and other alien objects.

6. The system as claimed in claim 5, wherein the medical implant, is at least one of a:
port-a-cath,
knee implant,
cardiac implant,
hip implant, and
dental implant.

7. The system as claimed in claim 6, wherein the system allows a user, enabling them to insert a syringe-needle into the port-a-Cath in a single-attempt, by assisting them with the port-a-cath location and orientation, that is disposed within a patient.

8. The system as claimed in claim 1, wherein the system has a transmitter-receiver unit that has a specific-bearing within, at least one of:
the see-through smart device, and a companion device.

9. The system as claimed in claim 8, wherein the companion devices are detachably attached to the see-through smart device; and
each of the companion device is capable of maintaining communication with the see-through smart device, even when physically detached from the said see-through smart device.

10. The system as claimed in claim 9, wherein the companion device, has a plurality of portions, that are detachably attached at different predefined locations on the see-through smart device, in order to gain the most optimal perspective.

11. The system as claimed in claim 8, wherein the transmitter-receiver unit is calibrated to detect foreign entities, that have varying sizes, shapes, textures, orientation and materials.

12. The system as claimed in claim 8, wherein the transmitter-receiver unit is operated in multiple modes based on the scenario, including continuous scan mode and pulse mode, in order to optimize power consumption and detection performance in real-time.

13. The system as claimed in claim 1, wherein the see-through smart device consists of a camera.

14. The system as claimed in claim 9, wherein the companion device has a provision of temporarily attaching with a magnification/enhancement equipment, in order to improve the strength of the visualization.

15. The system as claimed in claim 14, wherein the see-through smart device is at least one of: an AR glasses, an AR headset, a head-mounted smart device, a smart phone and a smart contact lens.

16. The system as claimed in claim 1, wherein the see-through smart device, has a plurality of internal electronic components, that generate the processed information by employing a software application, in order to integrate the received data obtained from the transmitter-receiver unit, by overlaying the processed information onto a portion of the body of the biological entity, that is within the field-of-view of the user.

17. The system as claimed in claim 16, wherein the software application is integrated with machine learning and generative AI algorithms, in order to continually improve the detection and visualization of the foreign entities, over time.

18. The system as claimed in claim 16, wherein the internal electronic components is at least one of: a memory unit, a central processing unit, a graphics processing unit, a power storage unit, a modulation unit, an amplification unit, a communication unit, a secondary electronic component, transmitter-receiver unit and an electromechanical component.

19. The system as claimed in claim 1, wherein the see-through smart device is equipped with a voice-recognition capability, in order to allow hands-free operation by the user.

20. The system as claimed in claim 1, wherein the see-through smart device communicates with an external database, in order to selectively fetch-share real-time information, regarding each of the detected foreign entities, as well as, the biological entity.
